(19) United States
(12) Patent Application Publication
(10) Pub. No.: US 2021/0148885 A1
(43) Pub. Date: May 20, 2021

(54) Compact Multipore Array with Embedded Electrodes for Sample Analysis

(71) Applicant: Nooma Bio, Inc., Santa Cruz, CA (US)

(72) Inventors: William B. Dunbar, Santa Cruz, CA (US); Xu Liu, Santa Cruz, CA (US); Philip Edward Zimny, Santa Cruz, CA (US); John Wallace Parce, Palo Alto, CA (US)

(21) Appl. No.: 16/951,974

(22) Filed: Nov. 18, 2020

Related U.S. Application Data
(60) Provisional application No. 62/937,047, filed on Nov. 18, 2019.
(60) Provisional application No. 62/962,857, filed on Jan. 17, 2020.

(51) Int. Cl. G01N 33/487

(52) U.S. Cl. CPC G01N 33/48721; G01N 33/48792

(57) ABSTRACT

The present disclosure includes a nanopore array devices that include a chip with an array of nanopore components for performing high-throughput and multiplexed assays. Aspects of the present disclosure include methods of screening drug targets and performing multiplexed assays using the nanopore chip of the devices and systems described in the present disclosure. Aspects of the present disclosure further include methods for performing single cell analysis using the devices and systems of the present disclosure.

### CROSS-REFERENCE TO RELATED APPLICATIONS

This application claims the benefit of U.S. Provisional Application Nos. 62/937,047, filed Nov. 18, 2019, and 62/962,857, filed Jan. 17, 2020, each of which is hereby incorporated by reference in its entirety.

### INTRODUCTION

Sample analysis systems are useful in performing a wide variety of analyses with useful applications across diverse disciplines. Current systems, however, are limited in their ability to efficiently and accurately process a large number of samples, for high-throughput detection and typically difficult to manufacture in a compact manner.

There is a need to maximize unit density and improve performance and cost of nanopore devices for high throughput detection of molecules.

### SUMMARY

Embodiments of system(s) for sample analysis include a set of nanopores and one or more embedded electrodes for providing electric fields relative to the set of nanopores and other structures (e.g., channels) of the system. In examples, the system is configured to process nucleic acid material at a throughput of 6 gigagases in less than 10 hours (or better), in a compact design having built-in redundancy. The system can include a substrate having a multi-layer configuration for defining the set of nanopores and other structures, and for positioning the set of electrodes.

In embodiments, the system can provide novel features at both chip and device levels, with onboard and remote electronic elements, and onboard microfluidic elements (e.g., in relation to fluid channels, in relation to handling of droplets and other forms of material, etc.) defined in layers of the system.

The embodiments of the system(s) described enable method(s) for rapid analysis of sample material for various multiplexed assays.

Aspects of the present disclosure include a nanopore array device for multiplexed assays, comprising: a chip comprising an array of nanopore components, wherein each nanopore component comprises: (a) a first pore positioned between, and fluidically connecting, a chamber and a first fluidic channel; (b) a second pore positioned between, and fluidically connecting, the chamber and a second fluidic channel; wherein the first pore and the second pore are spaced apart from each other by a distance; (c) one or more electrodes positioned within the first and second fluidic channels, wherein the one or more electrodes are configured to apply a first voltage across the first pore, and a second voltage across the second pore; and (d) one or more sensors configured to detect: a current measurement that detects capture and partial or full translocation of the molecule into and through the first pore; and a current measurement that detects capture and partial or full translocation of the molecule into and through the second pore.

In some embodiments, the chamber comprises one or more electrodes positioned within the chamber. In some embodiments, the one or more electrodes positioned within the first and second fluidic channels is connected to one or more of: an application-specific integrated circuit (ASIC), a field programmable gate array (FPGA), a microprocessor, and a signal processor. In some embodiments, the one or more electrodes positioned within the chamber is connected to one or more of: an application-specific integrated circuit (ASIC), a field programmable gate array (FPGA), a microprocessor, and a signal processor.

In some embodiments, the assay is performed on each of the nanopore components to evaluate ion conductance of each molecule in the nanopore components.

In some embodiments, the sensor is configured to detect: a voltage between the said electrode within the first fluidic channel and said electrode within the chamber for each nanopore components within the array simultaneously; and a voltage between the said electrode within the second fluidic channel and said electrode within the chamber for each nanopore component within the array simultaneously.

In some embodiments, the device comprises a processor configured to: determine from the sensor, the simultaneous presence of the molecule in both pores, and responsive to that determination, to adjust one or more of the first and second voltages to produce a first force and an opposing second force acting on the molecule, wherein the first and second forces control the direction and speed of the molecule translocating through the first and second pores.

In some embodiments, the array comprises 10 or more, 15 or more, 20 or more, 25 or more, 30 or more, 35 or more, 40 or more, or 50 or more arrays of nanopore components. In some embodiments, the arrays are arranged in parallel. In some embodiments, the arrays are arranged in series. In some embodiments, the array comprises 10×10 chips arranged on the device.

In some embodiments, the distance between an outermost edge or opening of the first fluidic channel and an outermost edge or opening of the second channel ranges from 200 μm to 5 mm. In some embodiments, the first fluidic channel has an inlet comprising a first opening on one end of the first fluidic channel and an outlet comprising a second opening on an opposite end of the first fluidic channel.

In some embodiments, the second fluidic channel has an inlet comprising a first opening at one end of the second fluidic channel and an outlet comprising a second opening on an opposite end of the second fluidic channel. In some embodiments, the inlet and outlet of the first and second fluidic channels is configured for fluidic filling or removal, and electrode access.

In some embodiments, the distance between an outermost edge of the first opening of the first fluidic channel and an outermost edge of the first opening of the second fluidic channel ranges from 100 μm to 500 μm. In some embodiments, the distance between an outermost edge of the second opening of the first fluidic channel and an outermost edge of the second opening of the second fluidic channel ranges from 100 μm to 500 μm. In some embodiments, the distance between the first pore and the second pore ranges from 10 nm to 5,000 nm. In some embodiments, the distance between the first pore and the second pore ranges from 300 nm to 2,000 nm.

In some embodiments, the dimensions of each of the nanopore components comprise a length of 8 mm and a width of 8 mm. In some embodiments, the dimensions of each of the nanopore components comprises a length of 5 mm and a width of 5 mm.

In some embodiments, the device further comprises a ground electrode located outside of the first and second fluidic channels.

In some embodiments, the first and second fluidic channels are sealed. In some embodiments, the first and second pores are sealed.

In some embodiments, the first and second pores have a depth ranging from about 0.5 nm to about 200 nm. In some embodiments, the first and second pores have a diameter ranging from about 0.5 nm to about 200 nm. In some embodiments, the depth of the first and second fluidic channel is about 1.5 μm. In some embodiments, the width of the first and second fluidic channel ranges from 50 to 500 μm. In some embodiments, the length of the first and second fluidic channel ranges from about 0.05 mm to about 4 mm.

In some embodiments, the thickness of the one or more electrodes is about 1 μm. In some embodiments, the one or more electrodes in contact with each of the fluidic channels comprises an electrode embedded in each of the fluidic channels.

In some embodiments, the first and second fluidic channel have a shape selected from: V-shape, U-shape, a square, rectangular, triangular, oval, hexangular, a cylindrical shape, and a polygon shape.

In some embodiments, the width of each of the first and second fluidic channel is 10 μm. In some embodiments, the first fluidic channel and the second fluidic channel have a V-shape. In some embodiments, the first fluidic channel and the second fluidic channel have openings on either end of the V-shape, wherein the V-shapes of the first and second fluidic channels arranged on the chip opposite one another with points of the V-shapes being adjacent to each other, and wherein the first nanopore is positioned at the point of the V-shape of the first fluidic channel and the second nanopore is positioned at the point of the V-shape of the second fluidic channel.

In some embodiments, the one or more electrodes are connected to a power supply configured to provide a first voltage between the first fluidic channel and the chamber and provide a second voltage between the chamber and the second fluidic channel.

Aspects of the present disclosure include a nanopore system for performing a multiplexed assay, comprising: a chip comprising array of nanopore components, wherein each nanopore component comprises: (a) a first pore positioned between, and fluidically connecting, a chamber and a first fluidic channel; (b) a second pore positioned between, and fluidically connecting, the chamber and a second fluidic channel; wherein the first pore and the second pore are spaced apart from each other by a distance; (c) one or more electrodes positioned within the first and second fluidic channels, wherein the one or more electrodes are configured to apply a first voltage across the first pore, and a second voltage across the second pore; and (d) a sensor subsystem configured to detect: a current measurement that detects capture and partial or full translocation of the molecule into and through the first pore; and a current measurement that detects capture and partial or full translocation of the molecule into and through the second pore.

In some embodiments, the molecule is a cell. In some embodiments, the cell is selected from: a neuron, a muscle cell, a cardiac cell, or an oocyte. In some embodiments, the molecule comprises one or more ion channels. In some embodiments, the single cell is loaded at an inlet comprising a first opening on one end of the first fluidic channel.

In some embodiments, the system comprises an automated/robotic pipette for loading the cell in an access port on the nanopore component. In certain embodiments, the automated/robotic pipette is configured to pipette low volumes (e., nanoliters) containing the cell in an opening comprising an inlet in the first fluidic channel, the second fluidic channel, or the chamber.

In some embodiments, the single cell is loaded at an inlet comprising a first opening on one end of the second fluidic channel. In some embodiments, the single cell is loaded at an inlet comprising a first opening of the chamber.

In some embodiments, the system further comprises a ground electrode positioned between the first and second fluidic channels.

In some embodiments, the sensor subsystem comprises a first sensor capable of identifying the presence of the molecule in the first pore. In some embodiments, the sensor subsystem comprises a second sensor capable of identifying the presence of the molecule in the second pore.

In some embodiments, the chamber comprises one or more electrodes positioned within the chamber.

In some embodiments, the system further comprises a processor and a computer-readable medium, comprising instructions that cause the processor to control the array of nanopore components as molecules translocate into and through the first and second pores of each nanopore component. In some embodiments, the processor comprises one or more of: an application-specific integrated circuit (ASIC), a field programmable gate array (FPGA), a microprocessor, and a signal processor. In some embodiments, the processor is connected to the one or more electrodes of the first and second fluidic channels. In some embodiments, the processor is connected to the one or more electrodes of the chamber.

In some embodiments, the assay is performed on each of the nanopore components to evaluate ion conductance of each molecule in the nanopore components.

In some embodiments, the sensor is configured to detect: a voltage between the said electrode within the first fluidic channel and said electrode within the chamber for each nanopore component within the array simultaneously; and a voltage between the said electrode within the second fluidic channel and said electrode within the chamber for each nanopore component within the array simultaneously.

In some embodiments, the device comprises a processor configured to: determine from the sensor, the simultaneous presence of the molecule in both pores, and responsive to that determination, to adjust one or more of the first and second voltages to produce a first force and an opposing second force acting on the molecule, wherein the first and second forces control the direction and speed of the molecule translocating through the first and second pores.

In some embodiments, the array comprises 10 or more, 15 or more, 20 or more, 25 or more, 30 or more, 35 or more, 40 or more, or 50 or more arrays of nanopore components.

In some embodiments, the arrays are arranged in parallel.

In some embodiments, the arrays are arranged in series.

In some embodiments, the array comprises 10×10 chips arranged on the device.

In some embodiments, the distance between an outermost edge or opening of the first fluidic channel and an outermost edge or opening of the second fluidic channel ranges from 200 μm to 5 mm.

In some embodiments, the first fluidic channel has an inlet comprising a first opening on one end of the first fluidic channel and an outlet comprising a second opening on an opposite end of the first fluidic channel.

In some embodiments, the second fluidic channel has an inlet comprising a first opening at one end of the second fluidic channel and an outlet comprising a second opening on an opposite end of the second fluidic channel. In some embodiments, the inlet and outlet of the first and second fluidic channels is configured for fluidic filling or removal, and electrode access. In some embodiments, the distance between an outermost edge of the first opening of the first fluidic channel and an outermost edge of the first opening of the second fluidic channel ranges from 100 μm to 500 μm. In some embodiments, the distance between an outermost edge of the second opening of the first fluidic channel and an outermost edge of the second opening of the second fluidic channel ranges from 100 μm to 500 μm. In some embodiments, the distance between the first pore and the second pore ranges from 10 nm to 5,000 nm. In some embodiments, the distance between the first pore and the second pore ranges from 300 nm to 2,000 nm.

In some embodiments, the dimensions of each of the nanopore components comprise a length of 8 mm and a width of 8 mm. In some embodiments, the dimensions of each of the nanopore components comprises a length of 5 mm and a width of 5 mm.

In some embodiments, the device further comprises a ground electrode located outside of the first and second fluidic channels. In some embodiments, the first and second fluidic channels are sealed. In some embodiments, the first and second pores are sealed.

In some embodiments, the first and second pores have a depth ranging from about 0.5 nm to about 200 nm. In some embodiments, the first and second pores have a diameter ranging from about 0.5 nm to about 200 nm. In some embodiments, the depth of the first and second fluidic channel is about 1.5 μm. In some embodiments, the width of the first and second fluidic channel ranges from 50 to 500 μm. In some embodiments, the length of the first and second fluidic channel ranges from about 0.05 mm to about 4 mm.

In some embodiments, the thickness of the one or more electrodes is about 1 μm. In some embodiments, the one or more electrodes in contact with each of the fluidic channels comprises an electrode embedded in each of the fluidic channels.

In some embodiments, the first and second fluidic channel have a shape selected from: V-shape, U-shape, a square, rectangular, triangular, oval, hexangular, a cylindrical shape, and a polygon shape.

In some embodiments, the width of each of the first and second fluidic channel is 10 μm. In some embodiments, the first fluidic channel and the second fluidic channel have a V-shape.

In some embodiments, the first fluidic channel and the second fluidic channel have openings on either end of the V-shape, wherein the V-shapes of the first and second fluidic channels arranged on the chip opposite one another with points of the V-shapes being adjacent to each other, and wherein the first nanopore is positioned at the point of the V-shape of the first fluidic channel and the second nanopore is positioned at the point of the V-shape of the second fluidic channel.

In some embodiments, the one or more electrodes are connected to a power supply configured to provide a first voltage between the first fluidic channel and the chamber, and provide a second voltage between the chamber and the second fluidic channel.

Aspects of the present disclosure include methods for controlling an array of nanopore components, the method comprising the steps of: (a) performing multiplexed assays on a chip comprising an array of nanopore components, wherein each nanopore component comprises: a first pore fluidically connected to a first fluidic channel; a second pore fluidically connected to a second fluidic channel; wherein the first pore and the second pore are spaced apart from each other by a distance; one or more electrodes positioned within the first and second fluidic channels, wherein the one or more electrodes are configured to apply a first voltage across the first pore, and a second voltage across the second pore; and one or more sensors configured to detect: a current measurement that detects capture and partial or full translocation of the molecule into and through the first pore; and a current measurement that detects capture and partial or full translocation of the molecule into and through the second pore. (b) controlling the array of nanopore components with a processor and a computer-readable medium comprising instructions, that cause the processor to: control the movement of charged molecules through the first and second pore in the array of nanopore components simultaneously, wherein each charged molecule translocates into and through the first and second pore of a single nanopore component on the array.

In some embodiments, the first pore is positioned between, and fluidically connecting, a chamber and the first fluidic channel. In some embodiments, the second pore is positioned between, and fluidically connecting, a chamber and the second fluidic channel.

In some embodiments, the charged molecule is a polypeptide.

In some embodiments, the charged molecule is a polynucleotide.

In some embodiments, the method further includes identifying a monomer unit of the molecule by measuring an ionic current across one of the nanopores when the monomer unit passes through the nanopore.

In some embodiments, the molecule is a cell. In some embodiments, cell is selected from: a neuron, a muscle cell, a cardiac cell, or an oocyte.

In some embodiments, the molecule comprises one or more ion channels.

In some embodiments, the processor is further configured to evaluate ion conductance of each molecule in the nanopore components.

In some embodiments, the monomer unit is selected from the group consisting of: a nucleotide, a nucleotide pair, and an amino acid residue. In some embodiments, the monomer unit is bound to a molecule. In some embodiments, the molecule is a DNA-binding protein.

In some embodiments, the DNA-binding protein is selected from the group consisting of: RecA, phase lambda repressor, NF-kB, and p53. In some embodiments, the polynucleotide is selected from the group consisting of: a double-stranded DNA, single-stranded DNA, double-stranded RNA, single-stranded RNA, and DNA-RNA hybrid. In some embodiments, the array of nanopore components are capable of identifying 10 or more, 20 or more, 30 or more, 40 or more, 50 or more, 60 or more, 70 or more, 80 or more, 90 or more, or 100 or more polymers.

Aspects of the present disclosure further include methods of fabricating a nanopore array device, the method comprising: (a) generating an electrode-supporting region, by: (i) depositing a first photoresist onto a substrate, (ii) patterning and etching a first channel, a second channel, and a first chamber onto the substrate, (iii) coating the entire surface of the substrate with a conductive material, and (iv) removing the first photo resist and conductive material on the surface of the substrate outside of the first channel, the second channel, and the first chamber; (b) generating a network of microchannels to form a buffer-supporting region in communication with first channel and the second channel, by: (i) depositing a second photoresist on the surface of the substrate to form a partially protected region of the first channel and the second channel, and to form a completely protected region of the first chamber, and (ii) patterning: a first microchannel that partially overlaps with the first channel, and a second microchannel that partially overlaps with the second channel, and removing the second photoresist to expose the first channel, the second channel, and the first chamber; and (c) sealing the electrode-supporting region and the buffer supporting region, by: (i) adhering a membrane layer to the exposed surface of the substrate to cover the first channel, the second channel, and the first chamber.

In some embodiments, the substrate is glass.

In some embodiments, the method further includes, before step (a)iv), coating the surface of the substrate with silicone dioxide, wherein said coating protects the conductive material from further patterning. In some embodiments, the method includes before step (a)iv), coating the surface of the substrate with thicker silicone dioxide, wherein said coating protects the conductive material from further patterning.

In some embodiments, the conductive material is an electrically conductive material.

In some embodiments, the electrically conductive material is silver.

In some embodiments, sealing the electrode-supporting region and the network of microchannels further comprises coating the membrane layer with a sealing material.

In some embodiments, the sealing material is a polymer.

In some embodiments, the polymer is selected from the group consisting of: polymethylmethacrylate (PMMA), polyethylene terephthalate (PETE), polycarbonate, and Polydimethylsiloxane (PDMS). In some embodiments, the sealing material is Polydimethylsiloxane (PDMS).

In some embodiments, the method further comprises patterning the sealing material.

In some embodiments, sealing the electrode-supporting region and the buffer-supporting region further comprises bonding a patterned sealing component onto the membrane layer.

In some embodiments, the method further comprises breaking the membrane layer to expose the first microchannel and the second microchannel.

In some embodiments, the method further comprises adding a buffer to the first microchannel and the second microchannel.

In some embodiments, the method further comprises sealing the first channel and the second channel after adding the buffer, with a sealing material.

In some embodiments, the first microchannel and the second microchannel each have a depth that is greater than the first channel and the second channel.

In some embodiments, the first chamber is always sealed with the membrane layer.

In some embodiments, the first channel and the second channel comprise one or more electrodes.

In some embodiments, the first channel and the second channel comprise one or more electrodes and a buffer. In some embodiments, the first microchannel and the second microchannel comprise a buffer.

In some embodiments, the first chamber comprises a ground electrode.

### BRIEF DESCRIPTION OF DRAWINGS

FIG. 1 depicts examples of a sample processing chip, and nanopore components within the processing chip, for sample analysis.

FIG. 2 depicts a first electrode design for implementation in an embodiment of the sample processing chip.

FIG. 3 depicts an example manufacturing method for the first electrode design shown in FIG. 2.

FIG. 4 depicts a second example manufacturing method for an electrode design shown in FIG. 2.

FIG. 5 depicts a first example fluidic design for implementation in an embodiment of the sample processing chip.

FIG. 6 depicts a first example fluidic sealing process for manufacturing an embodiment of the sample processing chip.

FIG. 7 depicts a first example device manufacturing process for manufacturing an embodiment of the sample processing chip.

FIG. 8 depicts an example device fluidic and electrode layout for an embodiment of the sample processing chip. In relation to FIG. 8, electrical and fluidic contacts are positioned toward the edges of the device for improved space efficiency. Furthermore, electrical wire contacts with fluidic channels are positioned at desired distances from respective nanopores. Furthermore, electrical wires and fluidic channels of the layout are embedded, in some variations of the schematic shown in FIG. 8. The system is also configured to provide robust bonding efficacy of respective device layers to each other, and to provide desired fluidic channel dimensions and curvatures to provide desired flow characteristics without clogging.

FIG. 9 depicts an example manufacturing method for an electrode design that includes an additional coating step.

FIG. 10 depicts an example manufacturing method for an electrode design.

FIG. 11 depicts an example of a sealing process for manufacturing an embodiment of the sample processing chip.

FIG. 12 depicts an example of a sealing process for manufacturing an embodiment of the sample processing chip.

FIG. 13 depicts a correlation between the fluidic channel length and channel resistance.

FIG. 14 depicts variations in the current, voltage, and resistance, showing that the system responds faster with a smaller resistance.

FIG. 15 depicts theoretical values and Clampex in the device.

FIG. 16 depicts electrode depletion.

FIG. 17 depicts an example channel geometry.

FIG. 18 depicts the correlation between channel resistance and channel width.

FIG. 19 depicts the correlation between channel resistance and channel width.

FIG. 20 depicts the time it takes a 5 mm device and a 8 mm device to drive DNA to the probe.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

The present disclosure provides devices and systems that include an array of nanopore devices. The present disclosure also provides methods using the array of nanopore devices. The array of nanopore components within the device provide high-throughput, multiplexed detection of molecules passing through the pores of the nanopore components.

I. Devices and Systems

Aspects of the present disclosure include a nanopore chip for multiplexed assays.

The nanopore chip of the present disclosure includes an array of nanopore components, wherein each nanopore component includes (a) a first pore positioned between, and fluidically connecting, a chamber and a first fluidic channel; (b) a second pore positioned between, and fluidically connecting, the chamber and a second fluidic channel; (c) one or more electrodes positioned within the first and second fluidic channels, wherein the one or more electrodes are configured to apply a first voltage across the first pore, and a second voltage across the second pore; and (d) one or more sensors configured to detect: a current measurement that detects capture and partial or full translocation of the molecule into and through the first pore; and a current measurement that detects capture and partial or full translocation of the molecule into and through the second pore.

Aspects of the present disclosure further include systems using the nanopore array device for performing multiplexed assays, the system including: a chip comprising an array of nanopore components, wherein each nanopore component comprises: (a) a first pore fluidically connected to a first fluidic channel; (b) a second pore fluidically connected to a second fluidic channel; (c) one or more electrodes positioned within the first and second fluidic channels, wherein the one or more electrodes are configured to apply a first voltage across the first pore, and a second voltage across the second pore; and (d) a sensor subsystem configured to detect: a current measurement that detects capture and partial or full translocation of the molecule into and through the first pore; and a current measurement that detects capture and partial or full translocation of the molecule into and through the second pore.

In some embodiments, each of the nanopore components in the array include a first fluidic channel and a second fluidic channel defined at a surface of an insulating substrate; a first pore fluidically connected to the first fluidic channel; a second pore fluidically connected to the second fluidic channel; one or more electrodes positioned within the first and second fluidic channels, wherein the one or more electrodes are configured to apply a first voltage across the first pore, and a second voltage across the second pore; and a sensor subsystem capable of identifying: the presence of a molecule during movement of the molecule through the first and second pore, wherein the sensor subsystem measures an ionic current across the first pore and second pores.

The nanopore array device of the present disclosure permits high-throughput detection of a large number of molecules with an array of nanopore components, minimizes crosstalk between common channels (e.g. chambers), lowers the resistance at the common ground with respect to the impedance of the capacitor at relevant frequency, and provides a sufficient depth of the channel containing one or more electrodes. In some embodiments, the fabrication process of making the nanopore array devices provides for higher patterning resolution and alignment, which decreases the capacitance by minimizing the center exposure area.

In some embodiments, each nanopore component includes at least one nanopore that forms an opening in a structure separating an interior space of the nanopore device into two volumes. The nanopore devices also includes at least a sensor in electrical communication with the opening and configured to identify objects (for example, by detecting changes in electrical signal parameters indicative of objects) passing through the nanopore. Nanopore components that can be used for the system and methods described herein are also disclosed in PCT Publication No. WO/2013/012881, and U.S. Pat. Nos. 9,863,912, and 10, 488, 394, which are hereby incorporated by reference in their entirety.

In some embodiments, the array comprises 10 or more, 15 or more, 20 or more, 25 or more, 30 or more, 35 or more, 40 or more, or 50 or more arrays of nanopore components. In some embodiments, the array comprises 10 or more, 20 or more, 30 or more, 40 or more, 50 or more, 60 or more, 70 or more, 80 or more, 90 or more, or 100 or more arrays of nanopore components. In some embodiments, 5 or more, 10 or more, 15 or more, 20 or more, 25 or more, or 30 or more arrays are arranged in parallel. In some embodiments, 5 or more, 10 or more, 15 or more, 20 or more, 25 or more, or 30 or more arrays are arranged in series. In some embodiments, the array includes 10 or more, 15 or more, 20 or more, 25 or more, 30 or more, 35 or more, 40 or more, or 50 or more arrays of nanopore components.

In some embodiments, the array comprises 5×5 chips arranged on the device. In some embodiments, the array comprises 5×5 chips arranged on the device, 6×6 chips arranged on the device, 7×7 chips arranged on the device, 8×8 chips arranged on the device, 9×9 chips arranged on the device, 11×11, 12×12 chips arranged on the device chips arranged on the device, 13×13 chips arranged on the device, 14×14 chips arranged on the device, or 15×15 chips arranged on the device. In some embodiments, the array comprises 10×10 chips arranged on the device. In certain embodiments, the arrays are arranged in parallel. In other embodiments, the arrays are arranged in series.

In some embodiments, the dimensions of each of the nanopore components comprise a length of 2 mm or more, 3 mm or more, 4 mm or more, 5 mm or more, 6 mm or more, 7 mm or more, 8 mm or more, 9 mm or more, of 8 mm or more, 9 mm or more, or 10 mm or more; and a width of 2 mm or more, 3 mm or more, 4 mm or more, 5 mm or more, 6 mm or more, 7 mm or more, 8 mm or more, 9 mm or more, of 8 mm or more, 9 mm or more, or 10 mm or more. In some embodiments, the dimensions of each of the nanopore components comprises a length of 2 mm and a width of 2 mm. In some embodiments, the dimensions of each of the nanopore components comprises a length of 3 mm and a width of 3 mm. In some embodiments, the dimensions of each of the nanopore components comprises a length of 4 mm and a width of 4 mm. In some embodiments, the dimensions of each of the nanopore components comprises a length of 5 mm and a width of 5 mm. In some embodiments, the dimensions of each of the nanopore components comprises a length of 8 mm and a width of 8 mm.

Nanopore Components

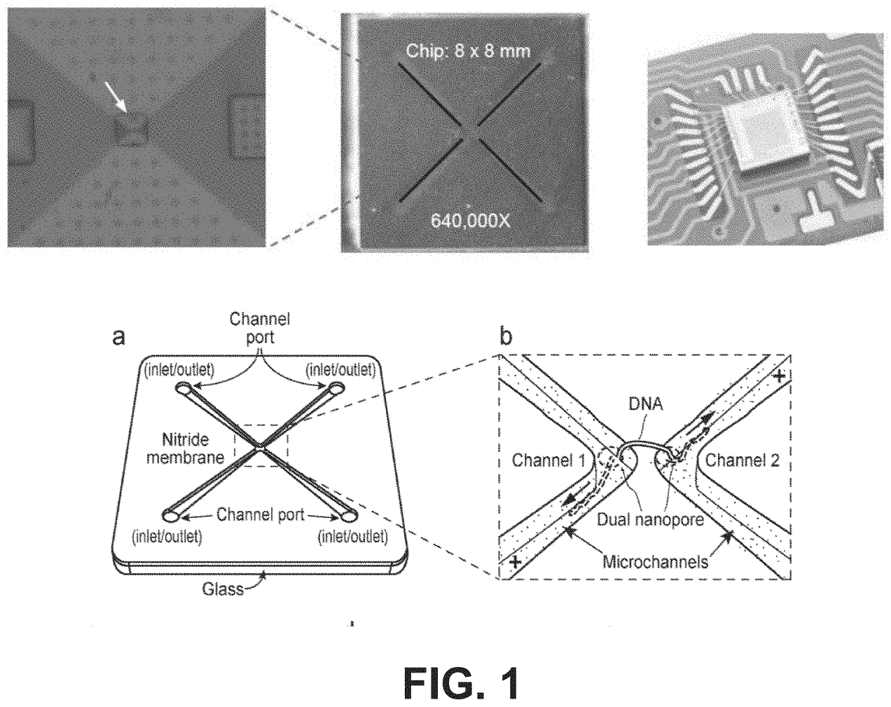

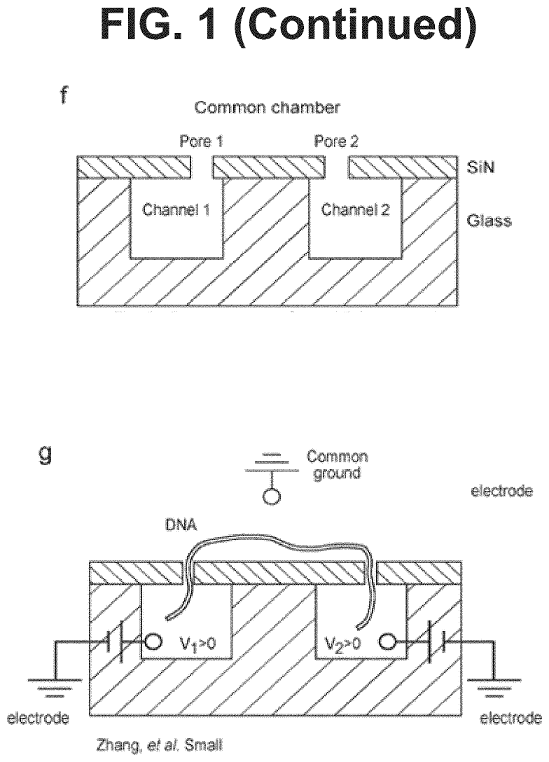

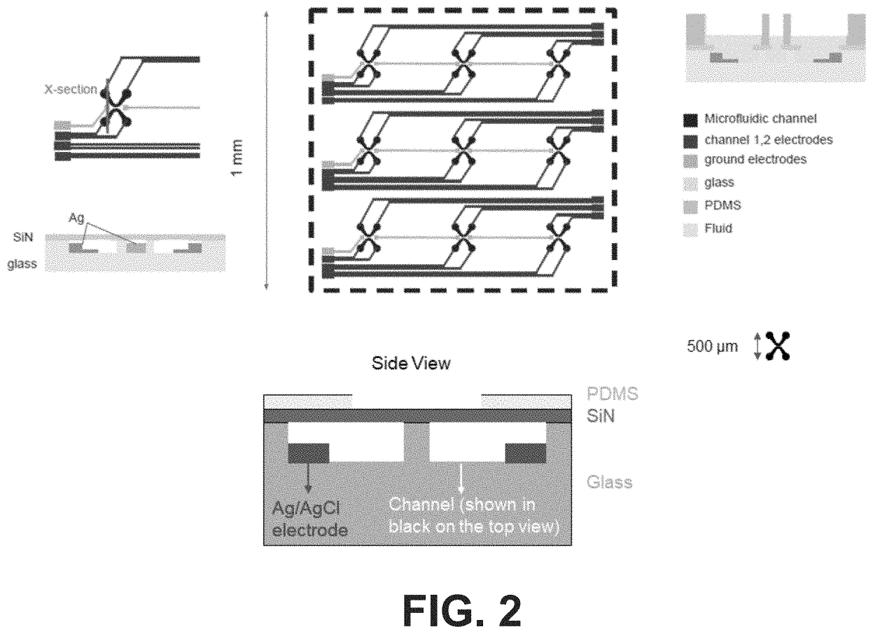

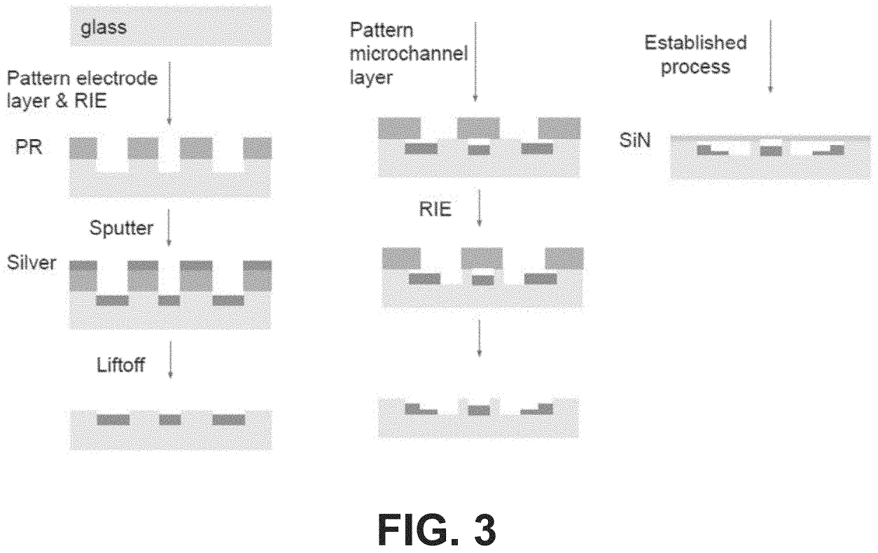

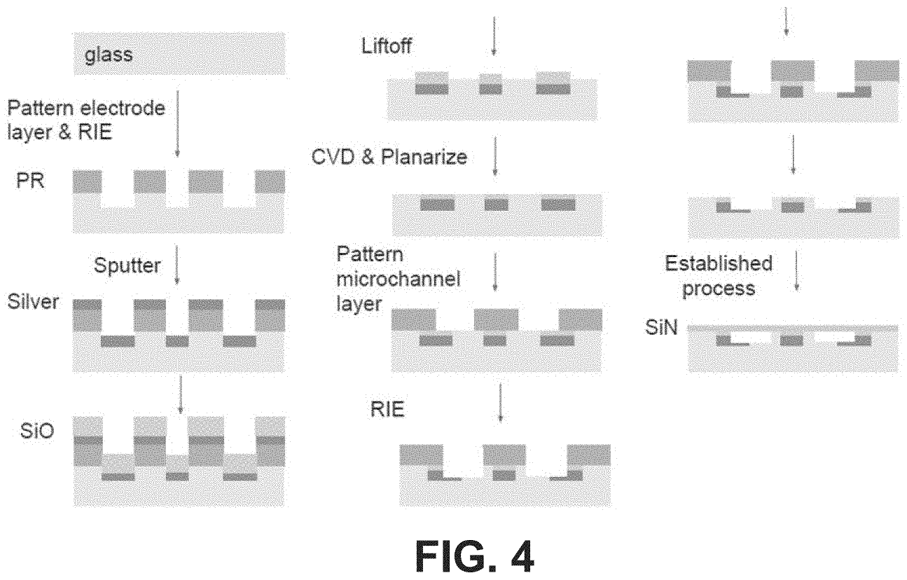

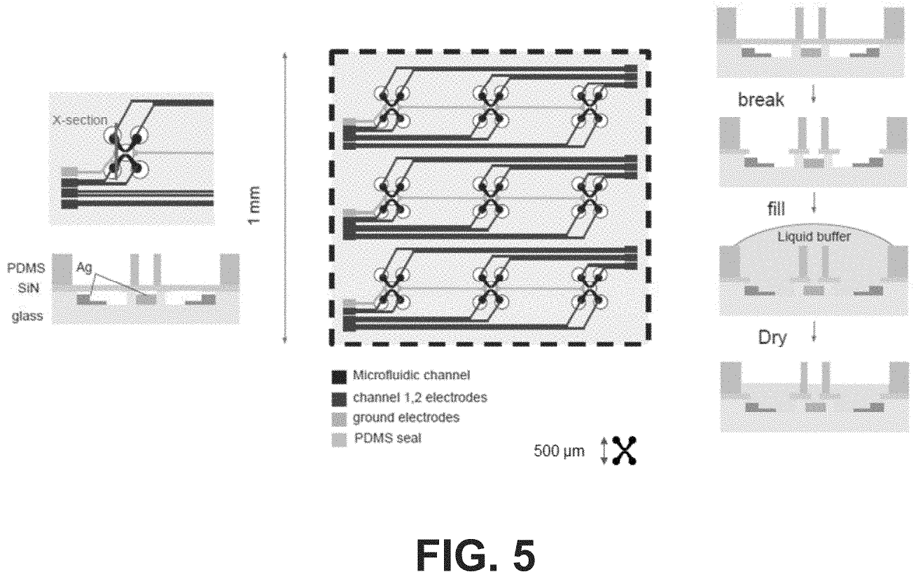

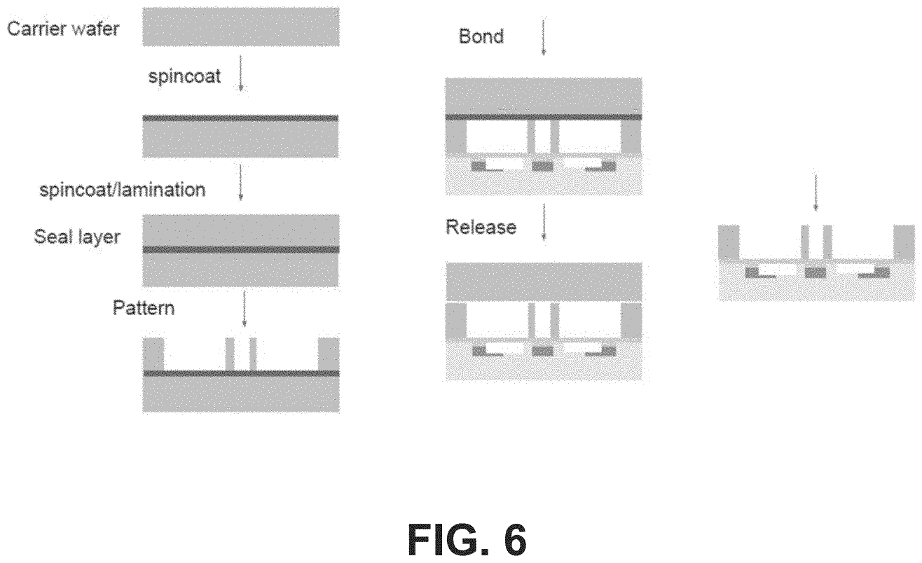

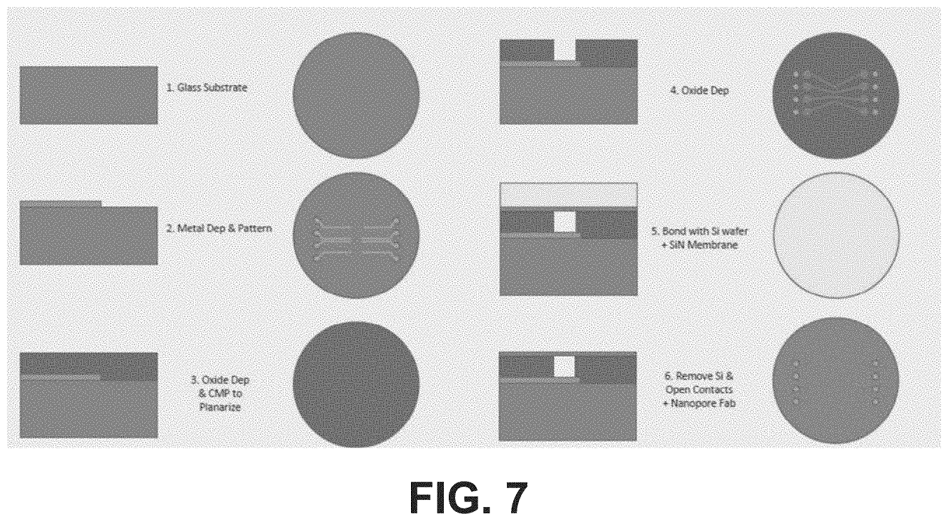

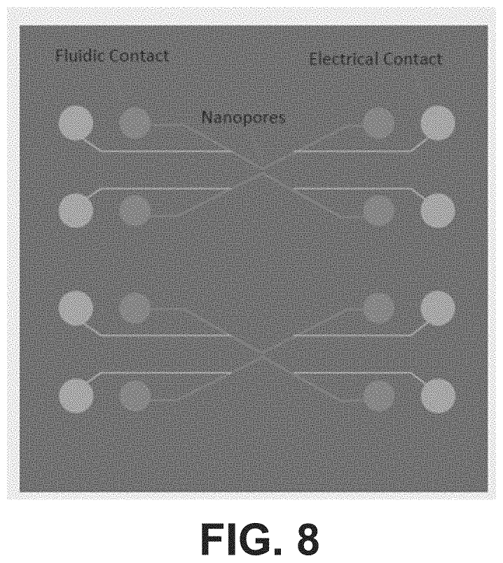

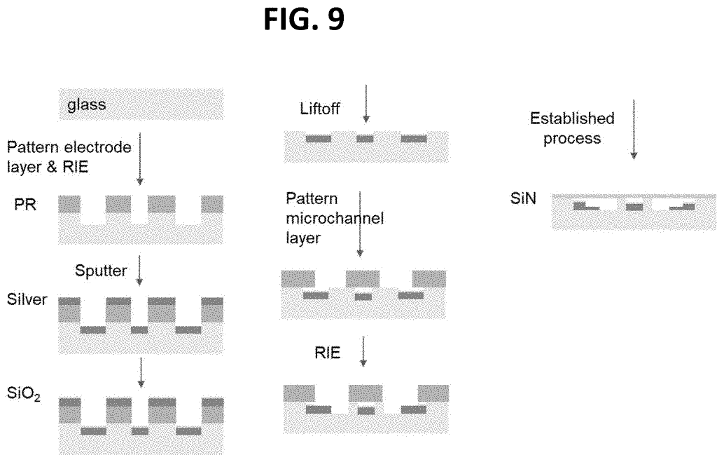

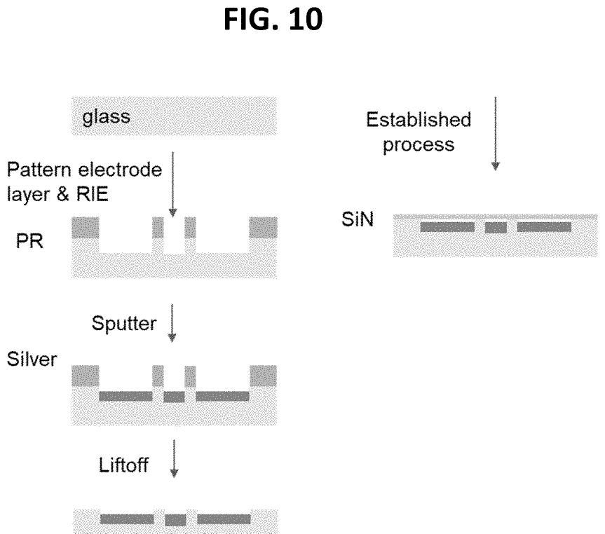

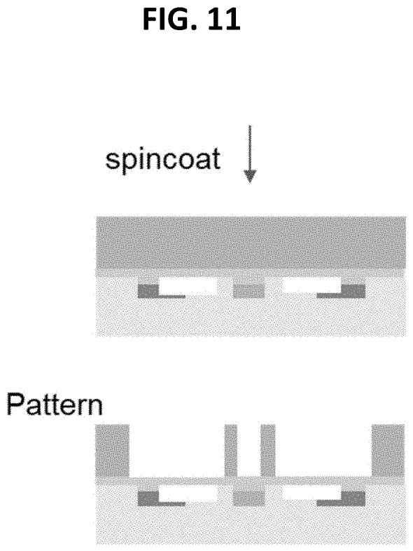

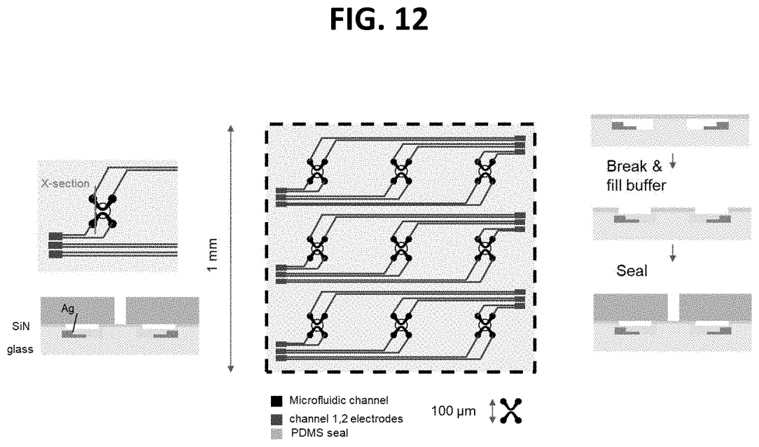

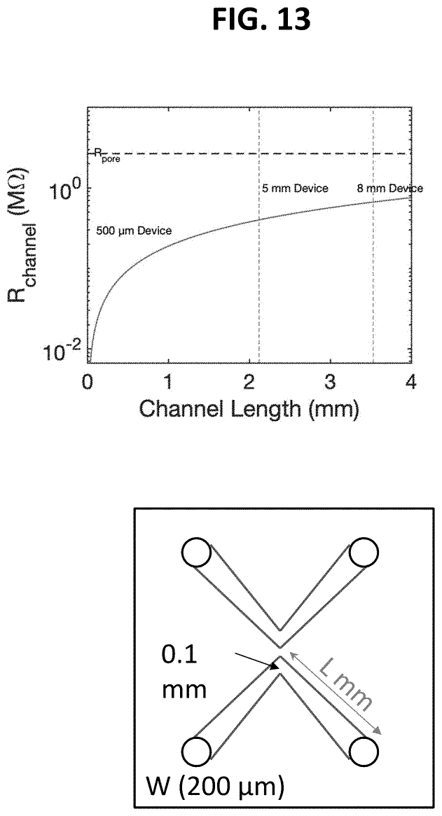

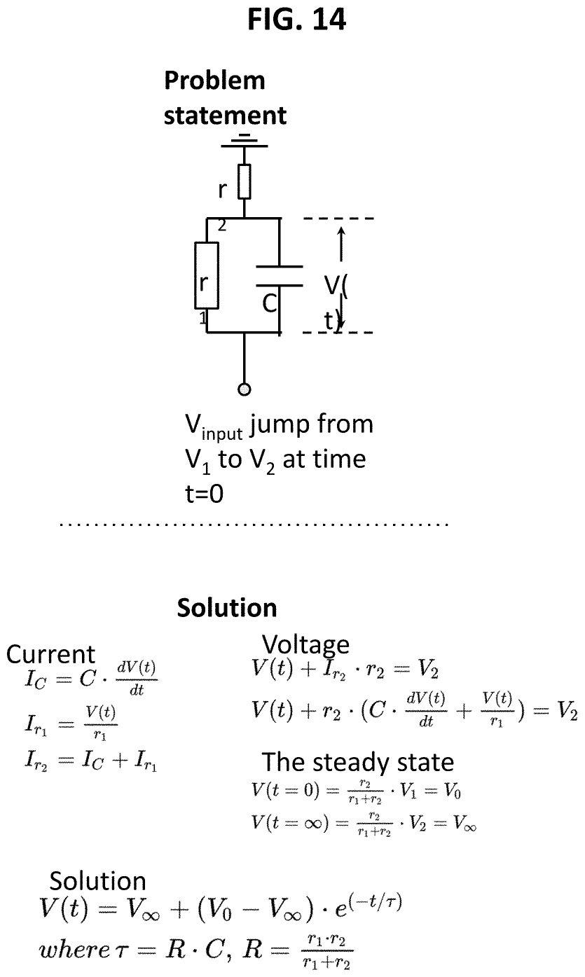

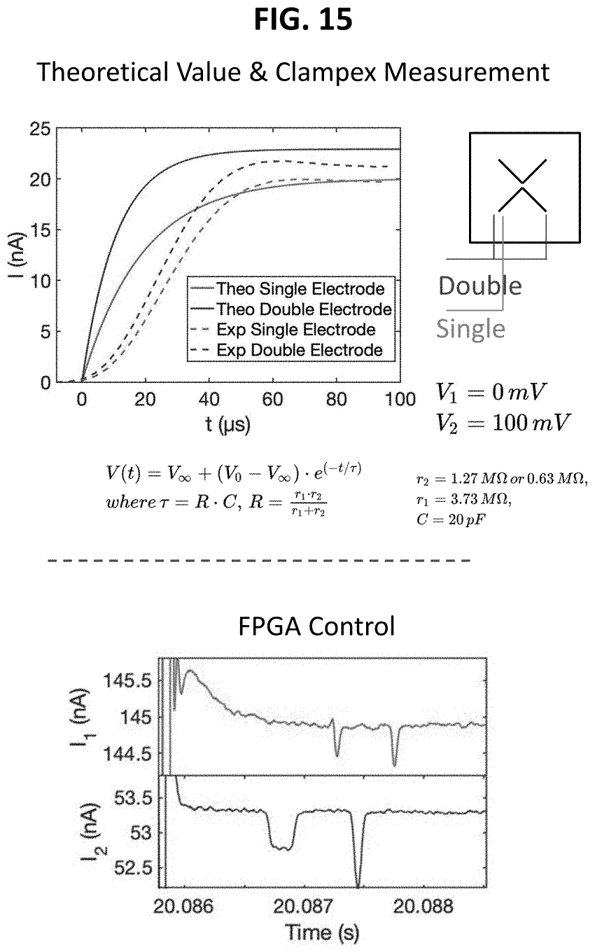

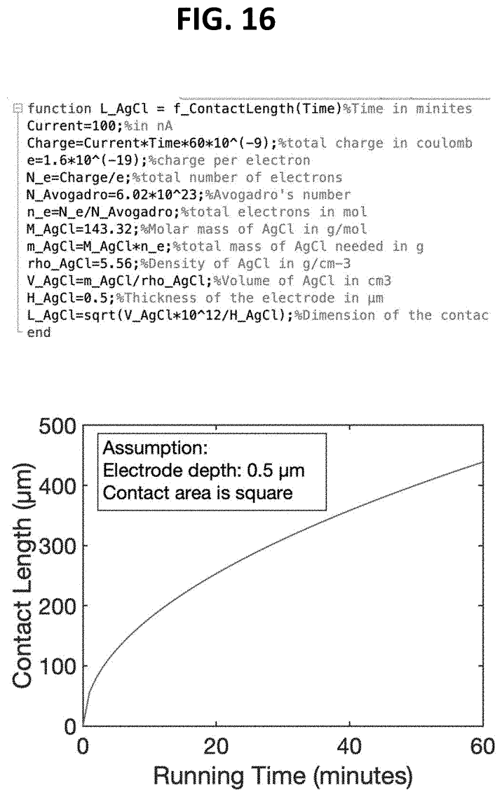

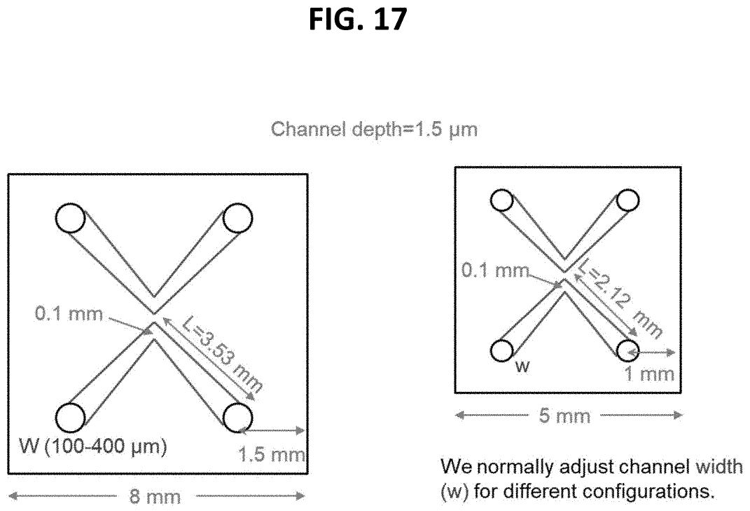

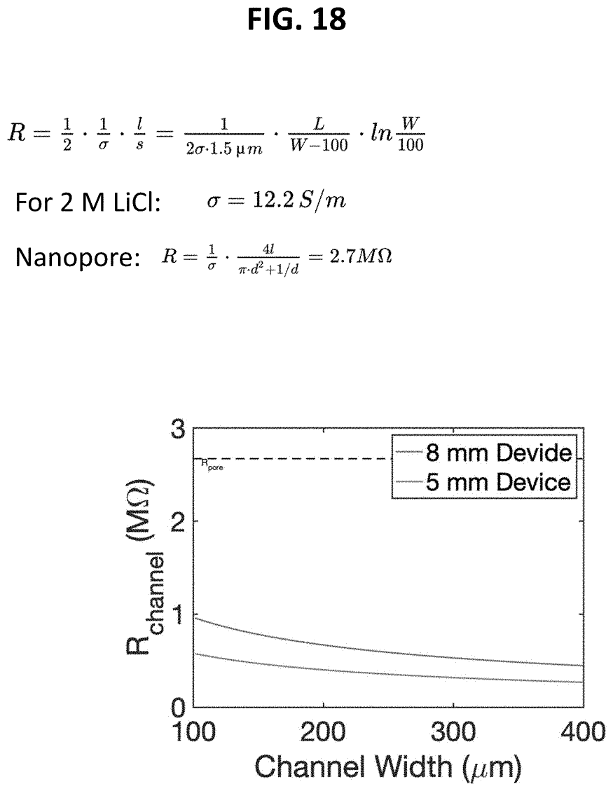

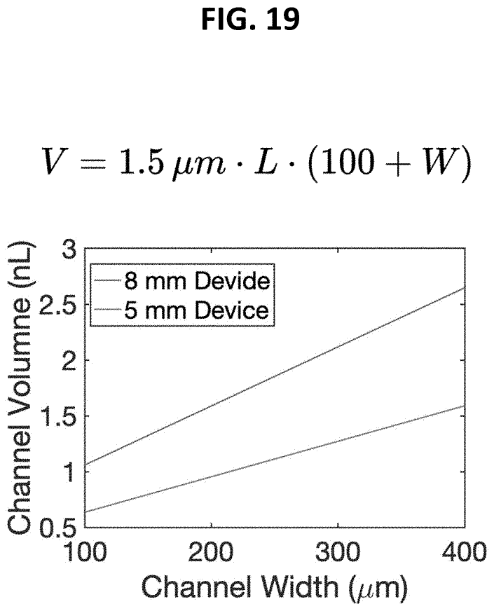

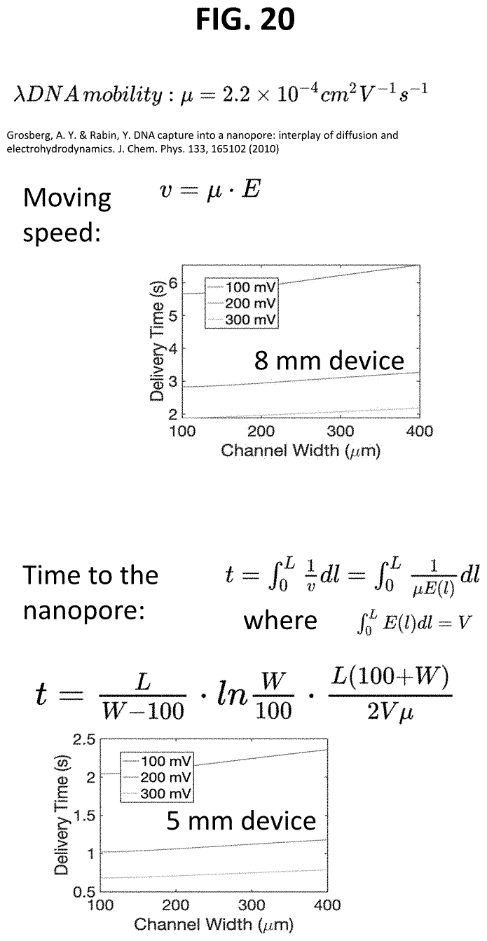

In some embodiments, the nanopore chip includes an array of nanopore components. Each nanopore component includes at least two nanopores (as shown in FIG. 1) that forms an opening in a structure separating an interior space of the nanopore component into two volumes. The nanopore devices also includes at least a sensor in electrical communication with the opening and configured to identify objects (for example, by detecting changes in electrical signal parameters indicative of objects) passing through the nanopore. Nanopore devices that may be used for the methods and systems described herein are also disclosed in PCT Publication Nos. WO/2013/012881 and WO/2018/236673, U.S. Application Publication No. 2017/0145481, and U.S. Pat. No. 9,863,912, which are hereby incorporated by reference in their entirety. Amplifiers and circuitry in the nanopore components that may be used for the methods and systems are also disclosed in U.S. Application Publication No. 2017/0145481, which is hereby incorporated by reference in its entirety.

In some embodiments, the nanopore(s) in the nanopore device(s) are nanoscale or microscale in relation to characteristic feature dimensions. In one aspect, each pore has a size that allows a small or large molecule (e.g., nucleic acid molecule or fragment) or microorganism to pass. In examples, nanopores can have a diameter from 1 nm through 100 nm; however, in variations of the examples, nanopores can have a diameter less than 1 nm or greater than 100 nm. In some embodiments, the diameter of the pores range from about 2 nm to about 50 nm. In some embodiments, the diameter of the pores is about 20 nm. In variations, a nanopore has a depth ranging from 1 nm to 10,000 nm; however, in other variations, a nanopore can have a depth less than 1 nm or greater than 10,000 nm. Furthermore, during an experimental run, nanopore dimensions may vary (within a suitable range), as described in further detail below.

Fluidic Channels

Aspects of the present disclosure include a chip comprising an array of nanopore components, each nanopore component comprises one or more fluidic channels (e.g. one or more chambers). In some embodiments, the one or more fluidic channels comprises a first fluidic channel, a chamber (e.g., middle fluidic channel) and a second fluidic channel. In some embodiments, the one or more channels comprises a first fluidic channel, a chamber, and a second fluidic channel, wherein the chamber is positioned between the first fluidic channel and the second fluidic channel. In some embodiments, the chip comprises a first nanopore providing communication between the first channel and a middle chamber (e.g., second channel).

In some embodiments, the device comprises a first fluidic channel. In some embodiments, the device comprises a middle chamber. As used herein, the term “middle chamber” is used interchangeably with the term “the chamber”. In some embodiments, the device comprises a second fluidic channel. In some embodiments, the fluidic channels of the nanopore component comprises one or more openings on a side opposite of the first and/or second pores. In some embodiments, the fluidic channels of the nanopore component comprises two openings on a side opposite of the first and/or second pores.

In some embodiments, the first fluidic channel has an inlet that includes a first opening on one end of the first fluidic channel and an outlet that includes a second opening on an opposite end of the first fluidic channel.

In some embodiments, the second fluidic channel has an inlet comprising a first opening at one end of the second fluidic channel and an outlet comprising a second opening on an opposite end of the second fluidic channel. In certain embodiments, the inlet and outlet of the first and second fluidic channels is configured for fluidic filling or removal, and electrode access.

In some embodiments, the first fluidic channel has a first opening on one end of and a second opening on an opposite end, where the second fluidic channel has a first opening at one end and a second opening on an opposite end. In some embodiments, the distance between an outermost edge of the first opening of the first fluidic channel and an outermost edge of the first opening of the second fluidic channel ranges from 50 μm to 1000 μm. In some embodiments, the distance between an outermost edge of the second opening of the first fluidic channel and an outermost edge of the second opening of the second fluidic channel ranges from 50 μm to 1000 μm. In some embodiments, the distance between an outermost edge of the first opening of the first fluidic channel and an outermost edge of the first opening of the second fluidic channel ranges from 100 μm to 500 μm. In some embodiments, the distance between an outermost edge of the second opening of the first fluidic channel and an outermost edge of the second opening of the second fluidic channel ranges from 100 μm to 500 μm.

In some embodiments, the first fluidic channel, the second fluidic channel, and/or the chamber contain one or more electrodes for connecting to a power supply so that a separate voltage can be established across each of the pores between the chambers. In some embodiments, the device comprises an electrode connected to a power supply configured to provide a first voltage between the first fluidic channel and the chamber of the device, and provides a second voltage between the chamber and a second fluidic channel of the device. In some embodiments, the chamber is positioned above the first and second pores. In some embodiments, the chamber is positioned above the first and second fluidic channels. In some embodiments, the chamber is positioned below the first and second pores. In some embodiments, the chamber is positioned between the first and second pores. In some embodiments, the chamber is positioned between the first and second fluidic channels. In some embodiments, the chamber is connected to a common ground relative to the first voltage.

In some embodiments, the width of the first and second fluidic channel ranges from 50-1000 μm. In some embodiments, the width of the first and second fluidic channel ranges from 50-500 μm. In some embodiments, the width of the first and second fluidic channel ranges from 50-100 μm, 100-150 μm, 150-200 μm, 200-250 μm, 250-300 μm, 300-350 μm, 350-400 μm, 450-500 μm, 500-550 μm, 550-600 μm, 600-650 μm, 650-700 μm, 700-750 μm, 750-800 μm, 800-850 μm, 850-900 μm, 900-950 μm, or 950-1000 μm.

In some embodiments, the length of the first and second fluidic channel ranges from about 0.005 mm to about 5 mm. In some embodiments, the length of the first and second fluidic channel ranges from about 0.05 mm to about 4 mm. In some embodiments, the length of the first and second fluidic channel ranges from about 0.0005 mm to about 4 mm. In some embodiments, the length of the first and second fluidic channel ranges from about 0.005 mm to about 0.05 mm. In some embodiments, the length of the first and second fluidic channel ranges from about 0.05 mm to about 0.5 mm. In some embodiments, the length of the first and second fluidic channel ranges from about 0.5 mm to about 1 mm. In some embodiments, the length of the first and second fluidic channel ranges from about 1 mm to about 1.5 mm. In some embodiments, the length of the first and second fluidic channel ranges from about 1.5 mm to about 2 mm. In some embodiments, the length of the first and second fluidic channel ranges from about 2 mm to about 2.5 mm. In some embodiments, the length of the first and second fluidic channel ranges from about 2.5 mm to about 3 mm. In some embodiments, the length of the first and second fluidic channel ranges from about 3 mm to about 3.5 mm. In some embodiments, the length of the first and second fluidic channel ranges from about 3.5 mm to about 4 mm. In some embodiments, the length of the first and second fluidic channel ranges from about 4.5 mm to about 5 mm.

In some embodiments, the first and second fluidic channel each have a depth ranging from 0.5 μm to 100 μm. In some embodiments, the first and second fluidic channel each have a depth ranging from 0.5 μm to 50 μm. In some embodiments, the first and second fluidic channel each have a depth ranging from 0.5 μm to 5 μm. In some embodiments, the first and second fluidic channel each have a depth ranging from 0.5 μm to 2 μm. In some embodiments, the first and second fluidic channel each have a depth ranging from 0.5 μm to 1.5 μm. In some embodiments, the first and second fluidic channel each have a depth of about 0.5 μm. In some embodiments, the first and second fluidic channel each have a depth of about 1 μm. In some embodiments, the first and second fluidic channel each have a depth of about 1.5 μm. In some embodiments, the first and second fluidic channel each have a depth of about 2 μm.

In some embodiments, the distance between an outermost edge or opening of the first channel and an outermost edge or opening of the second channel ranges from 50 μm to 5 mm. In some embodiments, the distance between an outermost edge or opening of the first channel and an outermost edge or opening of the second channel ranges from 200 μm to 5 mm. In some embodiments, the distance between an outermost edge or opening of the first channel and an outermost edge or opening of the second channel is about 50 μm, 100 μm, 200 μm, 300 μm, 400 μm, 500 μm, 600 μm, 700 μm, 800 μm, 900 μm, or 1000 μm. In some embodiments, the distance between an outermost edge of the first opening of the first fluidic channel and an outermost edge of the first opening of the second fluidic channel ranges from 100 μm to 500 μm. In some embodiments, the distance between an outermost edge or opening of the first channel and an outermost edge or opening of the second channel is about 1 mm, 2 mm, 3 mm, 4 mm, or 5 mm.

In some embodiments, the first and second fluidic channels are sealed. In some embodiments, the first and second fluidic channels are not sealed.

In some embodiments, the shape of the first and/or second fluidic channels can be circular, square, rectangular, hexagonal, triangular, oval, cylindrical, polygon, V-shape, U-shape, or any other suitable shape. In some embodiments, the first fluidic channel and the second fluidic channel each have a V-shape and each have openings on either end of the V-shape, the V-shapes of the first and second fluidic channels arranged on the chip opposite one another with points of the V-shapes being adjacent to each other, and wherein the first nanopore is positioned at the point of the V-shape of the first fluidic channel and the second nanopore is positioned at the point of the V-shape of the second fluidic channel. In some embodiments, each of the fluidic channels is a different shape. The fluidic channels are not limited to the shapes and/or sizes as described herein and can be any shape and/or size as required per conditions specified to its intended use.

Nanopores

Aspects of the present disclosure include one or more nanopores within each of the nanopore components. In some embodiments, each of the nanopore components comprises a first pore fluidically connected to a first fluidic channel. In some embodiments, each of the nanopore components comprises a second pore fluidically connected to a second fluidic channel. In some embodiments, the first pore and the second pore are spaced apart from each other by a distance. In some embodiments, the distance between the first pore and the second pore ranges from 100 nm to 1 μm. In some embodiments, the distance between the first pore and the second pore is about 100 nm, about 200 nm, about 300 nm, about 400 nm, about 500 nm, about 600 nm, about 700 nm, about 800 nm, about 900 nm, about 150 nm, about 250 nm, about 350 nm, about 450 nm, about 550 nm, or about 650 nm. In some embodiments, the distance between the first pore and the second pore ranges from about 100 nm to about 150 nm, about 150 nm to about 200 nm, about 200 nm to about 250 nm, about 250 nm to about 300 nm, about 300 nm to about 350 nm, about 350 nm to about 400 nm, about 400 nm to about 450 nm, about 450 to about 500 nm, about 500 to about 550 nm, 550 nm to about 600 nm, 600 nm to about 650 nm, 650 nm to about 700 nm, 700 nm to about 750 nm, 750 nm to about 800 nm, 800 nm to about 850 nm, 850 nm to about 900 nm, 900 nm to about 950 nm, or 950 nm to about 1000 nm.

In some embodiments, each of the pores in each nanopore component independently has a depth. In some embodiments, a first and second pore have a depth ranging from about 0.5 nm to about 200 nm. In some embodiments, each pore has a depth that is least about 0.3 nm. In some embodiments, each pore has a depth that is at least about 0.6 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, or 90 nm. In some embodiments, each pore has a depth that is no more than about 100 nm. Alternatively, the depth is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 or 10 nm. In some embodiments, the pore has a depth that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm. In some embodiments, the first pore has a depth of at least about 0.3 nm separating the first fluidic channel and the chamber and the second pore has a depth of at least about 0.3 nm separating the chamber and the second fluidic channel.

In some embodiments, each of the pores in the dual-pore chip independently has a depth. In one embodiment, each pore has a depth that is least about 0.3 μm. In some embodiments, each pore has a depth that is at least about 0.6 μm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm, 35 μm, 40 μm, 45 μm, 50 μm, 60 μm, 70 μm, 80 μm, or 90 μm. In some embodiments, each pore has a depth that is no more than about 100 μm. Alternatively, the depth is no more than about 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 or 10 μm. In some embodiments, the pore has a depth that is between about 1 μm and about 100 μm, or alternatively between about 2 μm and about 80 μm, or between about 3 μm and about 70 μm, or between about 4 μm and about 60 μm, or between about 5 μm and about 50 μm, or between about 10 μm and about 40 μm, or between about 15 μm and about 30 μm. In some embodiments, the depth of the first and second fluidic channel is about 1.5 μm. In some embodiments, the first pore has a depth of at least about 0.3 μm separating the first channel and the middle chamber and the second pore has a depth of at least about 0.3 μm separating the middle chamber and the second channel.

In some embodiments, each of the pores in each nanopore component independently has a size that allows a small or large molecule or microorganism to pass. In some embodiments, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 35 nm, 40 nm, 45 nm, 50 nm, 60 nm, 70 nm, 80 nm, 90 nm or 100 nm in diameter.

In some embodiments, a first and second pore of the nanopore component has a diameter ranging from about 0.5 nm to about 200 nm. In some embodiments, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm. In some embodiments, the first and second pores have a diameter ranging from about 20 nm to about 40 nm. In some embodiments, the first and second pores have a diameter ranging from about 1 nm to about 3 nm.

In some embodiments, the nanopore extends through a membrane. For example, the pore may be a protein channel inserted in a lipid bilayer membrane or it may be engineered by drilling, etching, or otherwise forming the pore through a solid-state substrate such as silicon dioxide, silicon nitride, grapheme, or layers formed of combinations of these or other materials.

In some embodiments, the first pore and the second pore in each nanopore component are spaced apart from each other by a distance. In some embodiments, nanopores of a nanopore component can be spaced apart at distances ranging from 5 nm-15,000 nm. In some embodiments, the nanopores of a nanopore component can be spaced apart at distances ranging from 10 nm to 1000 nm. However, in other variations, nanopores can be spaced apart less than 5 nm or greater than 15,000 nm. Furthermore, nanopores can be arranged in any position so long as they allow fluid communication between the chambers and have the prescribed size and distance between them. In some embodiments, the first pore and the second pore are about 10 nm to 500 nm apart from each other. In some embodiments, the first pore and the second pore are about 500 nm apart from each other. In one variation, the nanopores are placed so that there is no direct blockage between them. Still, in one aspect, the pores are substantially coaxial. In some embodiments, the distance between the first pore and the second pore ranges from 10 nm to 5,000 nm. In some embodiments, the distance between the first pore and the second pore ranges from 300 nm to 2,000 nm.

In some embodiments, the diameter of the pores of a nanopore component ranges from about 2 nm to about 50 nm. In some embodiments, the diameter of the pore is about 20 nm. In some embodiments, the diameter of the first and/or second pore ranges from about 2 nm to about 50 nm. In some embodiments, the diameter of the first and/or second pore ranges from about 2 nm to about 8 nm. In some embodiments, the diameter of the first and/or second pore ranges from about 10 nm to about 20 nm. In some embodiments, the diameter of the pore ranges from about 20 nm to about 30 nm. In some embodiments, the diameter of the first and/or second pore ranges from about 30 nm to about 40 nm. In some embodiments, the diameter of the first and/or second pore ranges from about 40 nm to about 50 nm. In some embodiments, the diameter of the first and/or second pore is about 2 nm, about 4 nm, about 6 nm, about 8 nm, about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm. In some embodiments, the diameter of the first and/or second pore is about 19 nm. In some embodiments, the first pore and the second pore have the same diameters. In some embodiments, the diameter of the first and/or second pore is about 21 nm. In some embodiments, the diameter of the first and/or second pore is about 22 nm. In some embodiments, the diameter of the first and/or second pore is about 23 nm. In some embodiments, the diameter of the first and/or second pore is about 24 nm. In some embodiments, the diameter of the first and/or second pore is about 25 nm. In some embodiments, the diameter of the first and/or second pore is about 27 nm. In some embodiments, the diameter of the first and/or second pore is about 29 nm. In some embodiments, the first pore and the second pore have different diameters. In some embodiments, the diameter of the pore is about 20 nm.

In some embodiments, the first pore and the second pore are about 10 nm to about 2 μm apart from each other.

In some embodiments, the diameter of the pores ranges from about 2 nm to about 50 nm. In some embodiments, the diameter of the pore is about 20 nm. In some embodiments, the diameter of the first and/or second pore ranges from about 2 nm to about 50 nm. In some embodiments, the diameter of the first and/or second pore ranges from about 2 nm to about 8 nm. In some embodiments, the diameter of the first and/or second pore ranges from about 10 nm to about 20 nm. In some embodiments, the diameter of the pore ranges from about 20 nm to about 30 nm. In some embodiments, the diameter of the first and/or second pore ranges from about 30 nm to about 40 nm. In some embodiments, the diameter of the first and/or second pore ranges from about 40 nm to about 50 nm. In some embodiments, the diameter of the first and/or second pore is about 2 nm, about 4 nm, about 6 nm, about 8 nm, about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm. In some embodiments, the diameter of the first and/or second pore is about 19 nm. In some embodiments, the first pore and the second pore have the same diameters. In some embodiments, the diameter of the first and/or second pore is about 21 nm. In some embodiments, the diameter of the first and/or second pore is about 22 nm. In some embodiments, the diameter of the first and/or second pore is about 23 nm. In some embodiments, the diameter of the first and/or second pore is about 24 nm. In some embodiments, the diameter of the first and/or second pore is about 25 nm. In some embodiments, the diameter of the first and/or second pore is about 27 nm. In some embodiments, the diameter of the first and/or second pore is about 29 nm. In some embodiments, the first pore and the second pore have different diameters. In some embodiments, the diameter of the pore is about 20 nm.

In some embodiments, the first pore and the second pore are about 500 nm apart from each other. In some embodiments, the first pore has a depth of at least about 0.3 nm separating the first fluidic channel and the chamber and the second pore has a depth of at least about 0.3 nm separating the chamber and the second channel. In some embodiments, the chamber is connected to a common ground relative to the two voltages. In some embodiments, the first and second pore have a depth ranging from about 3 nm to about 100 nm. In some embodiments, the first and second pores have a depth ranging from about 1 nm to about 3 nm.

In some embodiments, a nanopore in each nanopore component has a substantially round shape. “Substantially round”, as used here, refers to a shape that is at least about 80 or 90% in the form of a cylinder. However, in alternative embodiments, a nanopore component can include nanopores that are square, rectangular, triangular, oval, hexangular, a cylindrical shape, or of another morphology.

In some embodiments, the first and second pores are sealed prior to use. In some embodiments, the first and second pores are not sealed.

Electrodes

Aspects of the present disclosure include one or more electrodes configured to apply a first voltage across a first pore, and a second voltage across a second pore of a nanopore component. In some embodiments, one or more electrodes are positioned within each of the channels in the nanopore component. In some embodiments, the chip comprises a ground electrode. In some embodiments, the ground electrode is not positioned within any of the channels of the chip. In some embodiments, the ground electrode is positioned within one of the channels of the chip. In some embodiments, one or more electrodes are positioned within the first and second fluidic channels. In some embodiments, one or more electrodes are embedded into each of the channels in the nanopore component. In some embodiments, the one or more electrodes in contact with each of the fluidic channels comprises an electrode embedded in each of the fluidic channels.

In some embodiments, the chamber includes one or more electrodes positioned within the chamber.

In some embodiments, the chip comprises a ground electrode. In some embodiments, the ground electrode is not embedded into any of the channels of the chip. In some embodiments, the ground electrode is embedded into one of the channels of the chip. In some embodiments, one or more electrodes are embedded into the first and second fluidic channels. In some embodiments, when the 5 or more, 10 or more, 15 or more, 20 or more, 25 or more, or 30 or more arrays are arranged in series, one or more channels in each of the nanopore components share the same ground electrode. In some embodiments, when the 5 or more, 10 or more, 15 or more, 20 or more, 25 or more, or 30 or more arrays are arranged in parallel, one or more channels in each of the nanopore components share the same ground electrode. In some embodiments, a portion of the array of nanopore components in series share the same ground electrode. In some embodiments, a portion of the array of nanopore components in parallel share the same ground electrode. In some embodiments, the ground electrode is located outside of the first and second fluidic channels.

In some embodiments, the thickness of the electrode ranges from about 0.05 μm to about 2 μm. In some embodiments, the thickness of the electrode is about 0.5 μm. In some embodiments, the thickness of the electrode is about 1 μm. In some embodiments, the thickness of the electrode is about 1.5 μm. In some embodiments, the thickness of the electrode is about 1 μm. In some embodiments, the thickness of the electrode is about 2 μm. In some embodiments, the thickness of the electrode is about 1 μm. In some embodiments, the thickness of the electrode is about 3 μm.

In some embodiments, the nanopore component has electrodes positioned in the fluidic channels, geometrically constrained volume, or chambers and coupled to one or more power supplies in order to apply voltages across the nanopore(s). In some embodiments, the power supply includes a voltage-clamp or a patch-clamp, which can supply a voltage across each pore and measure the current through each pore independently. In this aspect, the power supply and the electrode configuration can set the chamber to a common ground for both power supplies. As such each nanopore can have its own respective applied voltage.

Each nanopore component applies voltages V1 and V2 that are independently applied at a first pore and a second pore, respectively. Two currents (I1 and I2) are also independently measured at the pores.

In some embodiments, a first voltage V1 and a second voltage V2 of different nanopores of a nanopore component are independently adjustable. In one aspect, where multiple nanopores are connected by a chamber, the chamber can be adjusted to be a ground relative to the two voltages. In one aspect, the chamber comprises a medium for providing conductance between each of the pores and the electrode in the chamber. In one aspect, the chamber includes a medium for providing a resistance between each of the nanopores and the electrode in the chamber. Keeping such a resistance sufficiently small relative to the nanopore resistances is useful for decoupling the two voltages and currents across the pores, which is helpful for the independent adjustment of the voltages.

Adjustment of the voltages can be used to control the movement of charged particles in the chambers. For instance, when both voltages are set in the same polarity, a properly charged particle can be moved from the first fluidic channel to the chamber and to the second fluidic channel, or the other way around, sequentially. In some aspects, when the two voltages are set to opposite polarity, a charged particle can be moved from either the first fluidic channel or the second fluidic channel to the chamber and kept there.

The adjustment of the voltages in the device can be particularly useful for controlling the movement of a large molecule, such as a charged polymer, that is long enough to cross both pores at the same time. In such an aspect, the direction and the speed of the movement of the molecule can be controlled by the relative magnitude and polarity of the voltages as described below.

In some embodiments, the first initial voltage ranges from 0 mV to 1000 mV. In some embodiments, the first initial voltage ranges from 100-200 mV, 200-300 mV, 300-400 mV, 400-500 mV, 500-600 mV, 600-700 mV, 700-800 mV, 800-900 mV, 900-1000 mV, or 1000 or more mV. In some embodiments, the first initial voltage is 100 mV, 200 mV, 300 mV, 400 mV, 500 mV, 600 mV, 700 mV, 800 mV, 900 mV, or 1000 mV. In some embodiments, the second initial voltage ranges from 0 mV to 1000 mV. In some embodiments, the second initial voltage ranges from 100-200 mV, 200-300 mV, 300-400 mV, 400-500 mV, 500-600 mV, 600-700 mV, 700-800 mV, 800-900 mV, 900-1000 mV, or 1000 or more mV. In some embodiments, the second initial voltage is 100 mV, 200 mV, 300 mV, 400 mV, 500 mV, 600 mV, 700 mV, 800 mV, 900 mV, or 1000 mV.

In some embodiments, the first voltage is applied between the first fluidic channel and the chamber. In some embodiments, the nanopore component comprises at least one electrode positioned within the second fluidic channel, wherein the at least one electrode is configured to provide a third voltage at the at second pore.

In some embodiments, the nanopore component comprises dual-amplifier electronics configured for voltage control and current measurement at the first pore and the second pore.

In some embodiments, method further comprises, after detecting a second ionic current, adjusting the first voltage at the first pore and setting a third voltage at the second pore so that at least a portion of the polynucleotide moves through the first pore and the second pore in a third direction, the third direction being from the first pore to the second pore. In some embodiments, the third voltage is higher than the first voltage.

In some embodiments, wherein the first voltage is 0 mV. In some embodiments, the first voltage ranges from 0-1000 mV. In some embodiments, the first voltage, the second voltage, the third voltage range, and/or fourth voltage, each independently range from 0 mV to 1000 mV. In some embodiments, adjusting the first voltage, the second voltage, the third voltage and/or fourth voltage each independently range from 0-1000 mV.

Sensors

As discussed above, in various aspects, the nanopore device further includes one or more sensors that generate electrical signals corresponding to materials passing through a nanopore.

The sensors used in a nanopore device can include any sensor suitable for identifying a molecule bound or unbound to a payload molecule. For instance, a sensor can be configured to identify the molecule by measuring a current, a voltage, a pH value, an optical feature, or residence time associated with the polymer. In other aspects, the sensor may be configured to identify one or more individual components of the molecule or one or more components bound or attached to the molecule. The sensor may be formed of any component configured to detect a change in a measurable parameter where the change is indicative of the molecule, a component of the molecule, or a component bound or attached to the molecule. In one aspect, the sensor includes a pair of electrodes placed at two sides of a pore to measure an ionic current across the pore when a molecule or other entity, in particular a target molecule, moves through the pore. In certain aspects, the ionic current across the pore changes measurably when a target molecule segment passing through the pore is bound to a payload molecule. Such changes in current may vary in predictable, measurable ways corresponding with, for example, the presence, absence, and/or size of the target molecule present.

In one embodiments, the sensor comprises electrodes that apply voltage and are used to measure current across the nanopore. Translocations of molecules through the nanopore provides electrical impedance (Z) which affects current through the nanopore according to Ohm's Law, V=IZ, where V is voltage applied, I is current through the nanopore, and Z is impedance. Inversely, the conductance G=1/Z are monitored to signal and quantitate nanopore events. The result when a molecule translocates through a nanopore in an electrical field (e.g., under an applied voltage) is a current signature that may be correlated to the molecule passing through the nanopore upon further analysis of the current signal.

When residence time measurements from the current signature are used, the size of the component can be correlated to the specific component based on the length of time it takes to pass through the sensing device.

In one embodiment, a sensor is provided in the nanopore device that measures an optical feature of the molecule, a component (or unit) of the molecule, or a component bound or attached to the molecule. One example of such measurement includes the identification of an absorption band unique to a particular unit by infrared (or ultraviolet) spectroscopy.

In some embodiments, the sensor is an electric sensor. In some embodiments, the sensor detects a fluorescent signature. A radiation source at the outlet of the pore can be used to detect that signature. Non-limiting examples of sensor circuitry in the nanopore device can be found in PCT Application Publication No. WO/2018/236673, which is hereby incorporated by reference in its entirety.

In some embodiments, one or more sensors are configured to detect a voltage between the said electrode within the first fluidic channel and an electrode within the chamber for each nanopore component within the array simultaneously; and a voltage between the electrode within the second fluidic channel and the electrode within the chamber for each nanopore component within the array simultaneously. A molecule can be loaded into both pores by two voltages having the same direction. In this example, once the direction of the voltage applied at the first pore is inversed and the new voltage-induced force is slightly less, in magnitude, than the voltage-induced force applied at the second pore, the molecule can continue moving in the same direction, but at a markedly lower speed. In this respect, the amplifier supplying voltage across the second pore also measures current passing through the second pore, and the ionic current then determines the identification of a molecule that is passing through the pore, as the passing of each different feature of the molecule would give rise to a different current signature (e.g., based on shifts in the ionic current amplitude). In some embodiments, the current event signature of the molecule is distinguished as it is translocating through the nanopores from current event signatures of background molecules or fragments by its mean depth, maximum depth, number of depth levels, area of depth and duration, noise level, or combination thereof.

Processor

Aspects of the present devices and systems include a processor. In some embodiments, a processor is used where the processor is provided or loaded with instructions to perform the techniques described herein. In some embodiments, the technique is implemented as a computer program product which is embodied in a computer readable storage medium and comprises computer instructions.

In some embodiments, a non-transitory computer-readable medium comprising instructions cause the processor to: determine from the sensor the simultaneous presence of the molecule in both pores, and responsive to that determination, to adjust one or more of the first and second voltages to produce a first force and an opposing second force acting on the molecule, wherein the first and second forces control the direction and speed of the movement of the molecule through the first and second pores.

In some embodiments, the processor is a digital signal processor (DSP), or the like. In an alternative embodiment, for example, the digital processing device may be a network processor having multiple processors including a core unit and multiple micro engines. Additionally, the digital processing device may include any combination of general-purpose processing device(s) and special-purpose processing device(s). In some embodiments, the processor is a field programmable gate array (FPGA) or an application-specific integrated circuit (ASIC). In some embodiments, the processor is a controller, wherein the controller is a field programmable gate array (FPGA) or an application-specific integrated circuit (ASIC). In some embodiments, the controller is a microcontroller.

In some embodiments, the one or more electrodes positioned within the chamber of the nanopore component is connected to one or more of: an application-specific integrated circuit (ASIC), a field programmable gate array (FPGA), a microprocessor, and a signal processor. In some embodiments, the one or more electrodes positioned within the first and/or second fluidic channels of the nanopore component is connected to one or more of: an application-specific integrated circuit (ASIC), a field programmable gate array (FPGA), a microprocessor, and a signal processor.

In some embodiments, the non-transitory computer-readable medium comprising instructions that cause the processor to determine from the sensor, the simultaneous presence of the molecule in both pores, and responsive to that determination. In some embodiments, the non-transitory computer-readable medium comprising instructions that cause the processor to adjust one or more of the first and second voltages to produce a first force and an opposing second force acting on the molecule. In certain embodiments, the first and second forces control the direction and speed of the molecule translocating through the first and second pores.

In some embodiments, the FPGA or ASIC executes control logic to change the movement of the target molecule for each of the nanopore components; f) direction of the target molecule for each of the nanopore components; g) speed of the target molecule for each of the nanopore components; h) voltage of the first and second pore for each of the nanopore components; or i) a combination thereof.

In some embodiments, the controller is configured to control the direction of movement of the target molecule for each of the nanopore components. In some embodiments, the device further comprises instructions that cause the processor to compute the speed of a feature of the target molecule for each of the nanopore components from the time difference between detection of the feature in the first pore and the second pore, and the known distance between pore one and pore two.

In some embodiments, the device further comprises instructions that cause the processor to perform a frequency sweep of the target molecule for each of the nanopore components in a first direction, second direction, or both. In some embodiments, the device further comprises instructions that cause the processor to perform an amplitude sweep of the target molecule for each of the nanopore components in the first direction, second direction, or both. In some embodiments, the device further comprises instructions that cause the processor to adjust the speed of the target molecule for each of the nanopore components. In some embodiments, wherein the speed ranges from 1 base pair per millisecond to 10 base pairs per millisecond.

In some embodiments, the device further comprises instructions that cause the processor to evaluate ion conductance of each molecule in the nanopore component.

II. Methods

Aspects of the present disclosure include methods of controlling the movement of a molecule through a nanopore using the devices and systems described in the present disclosure. Aspects of the present disclosure further include methods of controlling an array of nanopore components. Aspects the present disclosure further include methods of fabricating a nanopore array device of the present disclosure.

In one aspect, the method for controlling an array of nanopore components includes the steps of: (a) performing multiplexed assays on a chip comprising an array of nanopore components, wherein each nanopore component comprises: a first pore fluidically connected to a first fluidic channel; a second pore fluidically connected to a second fluidic channel; one or more electrodes positioned within the first and second fluidic channels, wherein the one or more electrodes are configured to apply a first voltage across the first pore, and a second voltage across the second pore; and one or more sensors configured to detect: a current measurement that detects capture and partial or full translocation of the molecule into and through the first pore; and a current measurement that detects capture and partial or full translocation of the molecule into and through the second pore, (b) controlling the array of nanopore components with a processor and a computer-readable medium comprising instructions, that cause the processor to: control the movement of charged molecules into and through the first and second pore in the array of nanopore components simultaneously, wherein each charged molecule translocates into and through the first and second pore of a single nanopore component on the array.

In some embodiments, such methods can be used for high-throughput drug screening using the array of nanopore components. In some embodiments, such methods can be used for screening for drug targets.

In some embodiments, the method comprises loading the charged molecule into the nanopore component. In certain embodiments, the method comprises loading the charged molecule in the first fluidic channel, the second fluidic channel, or the chamber of the nanopore component. In certain embodiments, the method comprises loading the charged molecule in the first fluidic channel. In certain embodiments, the method comprises loading the charged molecule into the second fluidic channel. In certain embodiments, the method comprises loading the charged molecule in the chamber. In certain embodiments, the method comprises loading the charged molecule at the inlet or opening of the first fluidic channel, the second fluidic channel, or chamber of the nanopore component.

In some embodiments, loading the sample containing the molecule in the first fluidic channel, the second fluidic channel, or the chamber provides for exchange of different reagents.

In some embodiments, the method comprises lysing the molecule. In certain embodiments, lysing the molecule comprises contacting the molecule with one or more lysing reagents. In some embodiments, lysing the molecule comprises applying one or more voltages within the first fluidic channel, the second fluidic channel, and/or the chamber to lyse the molecule. In some embodiments, lysing the molecule comprises applying a pressure to the first fluidic channel, the second fluidic channel, and/or the chamber to lyse the molecule.

In some embodiments, the method comprises contacting the first fluidic channel, the second fluidic channel, and/or the chamber containing the molecule with one or more reagents to digest or breakdown molecule fragments (e.g. non-nucleic acid materials such as proteins, enzymes, and the like). In certain embodiments, the method comprises contacting the first fluidic channel, the second fluidic channel, and/or the chamber containing the molecule with one or more proteases. In certain embodiments, the protease is proteinase K. In some embodiments, contacting the first fluidic channel, the second fluidic channel, and/or the chamber containing the molecule with one or more reagents provides for translocating target molecules such as DNA or RNA through the nanopores within the nanopore components.

In some embodiments, loading comprises pipetting, with a robotic pipette, a volume containing single cell contents, into a port of the nanopore component. In certain embodiments, robotic pipettes that move small volumes (e.g., nL volumes) can deliver single cell contents, prepped in ultra-low volumes outside the chip, into the chip at an appropriate delivery small volume port. In certain embodiments, loading comprises pipetting, with a robotic pipette, a volume containing single cell contents, into an opening of the first fluidic channel, the second fluidic channel, or the chamber of the nanopore component. In some embodiments, the port of the nanopore component is fluidically connected to the first fluidic channel, the second fluidic channel or the chamber.

In some embodiments, the method further comprises measuring ion conductance of each molecule in the nanopore components.

In some embodiments, the molecule is a cell. In some embodiments, the cell is selected from: a neuron, a muscle cell, a cardiac cell, and an oocyte. In some embodiments, the molecule includes one or more ion channels within the cell.

In some embodiments, the molecule is a polypeptide.

In some embodiments, the molecule is a polynucleotide.

In some embodiments, the method further comprising identifying a monomer unit of the molecule by measuring an ionic current across one of the nanopores when the monomer unit passes through the nanopore.

In some embodiments, the monomer unit is selected from the group consisting of: a nucleotide, a nucleotide pair, and an amino acid residue.

In some embodiments, the monomer unit is bound to a molecule.

In some embodiments, the molecule is a DNA-binding protein. In some embodiments, the DNA-binding protein is selected from the group consisting of: RecA, phase lambda repressor, NF-kB, and p53.

In some embodiments, the molecule is a polynucleotide selected from the group consisting of: a double-stranded DNA, single-stranded DNA, double-stranded RNA, single-stranded RNA, and DNA-RNA hybrid. In some embodiments, the polynucleotide is bound to a payload molecule.

In some embodiments, the array of nanopore components are capable of identifying 10 or more, 20 or more, 30 or more, 40 or more, 50 or more, 60 or more, 70 or more, 80 or more, 90 or more, or 100 or more molecules.

In some embodiments, the molecule comprises one or more payload molecules bound to the molecule. In some embodiments, the molecule comprises one or more payload molecules hybridized to the molecule. In some embodiments, the molecule is selected from the group consisting of: a nucleic acid, a TALEN, a CRISPR, a peptide nucleic acid, and a chemical compound. In some embodiments, the one or more features comprises a DNA-binding protein, a polypeptide, an anti-DNA antibody, a streptavidin, a transcription factor, a histone, a peptide nucleic acid (PNA), a DNA-hairpin, a DNA molecule, an aptamer, or a combination thereof. In some embodiments, the molecule is a CRISPR/Guide RNA complex. In some embodiments, the molecule is a guide RNA. In some embodiments, the molecule is an mRNA. In some embodiments, the molecule is selected from: shRNA, miRNA, siRNA, and CRISPR guide RNA. In some embodiments, the molecule is shRNA. Methods for making and using CRISPR guide RNA are known and described in, for example, Larson, et al., Nature Protocols 8:2180-2196 (2013), U.S. Pat. No. 10,253,365, and U.S. Publication No. 2014/0068797, which are incorporated by reference herein in their entirety.

Non-limiting examples of payload molecules bound to the molecule such as a polynucleotide can be found in can be found in U.S. Patent Publication No. 2018/0023115, which is hereby incorporated by reference in its entirety. For example, a payload molecule can include a dendrimer, double stranded DNA, single stranded DNA, a DNA aptamer, a fluorophore, a protein, a polypeptide, a nanorod, a nanotube, fullerene, a PEG molecule, a liposome, or a cholesterol-DNA hybrid. In some embodiments, the polynucleotide and the payload are connected directly or indirectly via a covalent bond, a hydrogen bond, an ionic bond, a van der Waals force, a hydrophobic interaction, a cation-pi interaction, a planar stacking interaction, or a metallic bond. The payload adds size to the target polynucleotide or amplicon, and facilitates detection, with the amplicon bound to the payload having a markedly different current signature when passing through the nanopore than background molecules. In some embodiments, the payload molecule comprises an azide chemical handle for attachment to a primer. In some embodiments, the primer is bound to a biotin molecule. In some embodiments, the payload molecule can bind to another molecule to affect the bulkiness of the molecule, thereby enhancing the sensitivity of detection of the amplicon in a nanopore. In some embodiments, the primer is bound to or comprises a binding site for binding to a biotin molecule. In some embodiments, the biotin is further bound by streptavidin to increase the size of the payload molecule for enhanced detection in a nanopore over background molecules. The added bulk can produce a more distinct signature difference between amplicon comprising a target sequence and background molecules.

In this embodiment, attachment of a payload to a primer or amplicon can be achieved in a variety of ways. For example, the primer may be a dibenzocyclooctyne (DBCO) modified primer, effectively labeling all amplicons with a DBCO chemical group to be used for conjugation purposes via copper-free “click” chemistry to an azide-tagged amplicon or primer.

In some aspects, the primer comprises a chemical modification that causes or facilitates recognition and binding of a payload molecule. For example, methylated DNA sequences can be recognized by transcription factors, DNA methyltransferases or methylation repair enzymes. In other embodiments, biotin may be incorporated into, and recognized by, avidin family members. In such embodiments, biotin forms the fusion binding domain and avidin or an avidin family member is the polymer scaffold-binding domain on the fusion. Due to their binding complementarity, payload molecule binding domains on a primer/amplicon and primer binding domains on a payload molecule may be reversed so that the payload binding domain becomes the primer binding domain, and vice versa.

Molecules, in particular proteins, that are capable of specifically recognizing nucleotide binding motifs are known in the art. For instance, protein domains such as helix-turn-helix, a zinc finger, a leucine zipper, a winged helix, a winged helix turn helix, a helix-loop-helix and an HMG-box, are known to be able to bind to nucleotide sequences. Any of these molecules may act as a payload molecule binding to the amplicon or primer.

In some aspects, the payload binding domains can be locked nucleic acids (LNAs), bridged nucleic acids (BNA), Protein Nucleic Acids of all types (e.g. bisPNAs, gamma-PNAs), transcription activator-like effector nucleases (TALENs), clustered regularly interspaced short palindromic repeats (CRISPRs), or aptamers (e.g., DNA, RNA, protein, or combinations thereof).

In some aspects, the payload binding domains are one or more of DNA binding proteins (e.g., zinc finger proteins), antibody fragments (Fab), chemically synthesized binders (e.g., PNA, LNA, TALENS, or CRISPR), or a chemical modification (i.e., reactive moieties) in the synthetic polymer scaffold (e.g., thiolate, biotin, amines, carboxylates).

Aspects of the present methods include controlling the array of nanopore components with a processor. In some embodiments, a processor is used where the processor is provided or loaded with instructions to perform the techniques described herein. In some embodiments, the technique is implemented as a computer program product which is embodied in a computer readable storage medium and comprises computer instructions.

In some embodiments, a non-transitory computer-readable medium comprising instructions cause the processor to: determine from the sensor the simultaneous presence of the molecule in both pores, and responsive to that determination, to adjust one or more of the first and second voltages to produce a first force and an opposing second force acting on the molecule, wherein the first and second forces control the direction and speed of the movement of the molecule through the first and second pores.

In some embodiments, the processor is a digital signal processor (DSP), or the like. In an alternative embodiment, for example, the digital processing device may be a network processor having multiple processors including a core unit and multiple micro engines. Additionally, the digital processing device may include any combination of general-purpose processing device(s) and special-purpose processing device(s). In some embodiments, the processor is a field programmable gate array (FPGA) or an application-specific integrated circuit (ASIC). In some embodiments, the processor is a controller, wherein the controller is a field programmable gate array (FPGA) or an application-specific integrated circuit (ASIC). In some embodiments, the controller is a microcontroller.

In some embodiments, the non-transitory computer-readable medium comprising instructions that cause the processor to determine from the sensor, the simultaneous presence of the molecule in both pores, and responsive to that determination. In some embodiments, the non-transitory computer-readable medium comprising instructions that cause the processor to adjust one or more of the first and second voltages to produce a first force and an opposing second force acting on the molecule. In certain embodiments, the first and second forces control the direction and speed of the molecule translocating through the first and second pores.

In some embodiments, the FPGA or ASIC executes control logic to change the movement of the target molecule for each of the nanopore components; f) direction of the target molecule for each of the nanopore components; g) speed of the target molecule for each of the nanopore components; h) voltage of the first and second pore for each of the nanopore components; or i) a combination thereof.

In some embodiments, the controller is configured to control the direction of movement of the target molecule for each of the nanopore components. In some embodiments, the device further comprises instructions that cause the processor to compute the speed of a feature of the target molecule for each of the nanopore components from the time difference between detection of the feature in the first pore and the second pore, and the known distance between pore one and pore two.

In some embodiments, the device further comprises instructions that cause the processor to perform a frequency sweep of the target molecule for each of the nanopore components in a first direction, second direction, or both. In some embodiments, the device further comprises instructions that cause the processor to perform an amplitude sweep of the target molecule for each of the nanopore components in the first direction, second direction, or both. In some embodiments, the device further comprises instructions that cause the processor to adjust the speed of the target molecule for each of the nanopore components. In some embodiments, wherein the speed ranges from 1 base pair per millisecond to 10 base pairs per millisecond.

In some embodiments, the device further comprises instructions that cause the processor to evaluate ion conductance of each molecule in the nanopore components.

Aspects of the present disclosure further include methods of fabricating a nanopore array device. The method of fabricating a nanopore array device includes (a) generating an electrode-supporting region, by: (i) depositing a first photoresist onto a substrate, (ii) patterning and etching a first channel, a second channel, and a first chamber onto the substrate, (iii) coating the entire surface of the substrate with a conductive material, and (iv) removing the first photo resist and conductive material on the surface of the substrate outside of the first channel, the second channel, and the first chamber; (b) generating a network of microchannels to form a buffer-supporting region in communication with first channel and the second channel, by: (i) depositing a second photoresist on the surface of the substrate to form a partially protected region of the first channel and the second channel, and to form a completely protected region of the first chamber, and (ii) patterning: a first microchannel that partially overlaps with the first channel, and a second microchannel that partially overlaps with the second channel, and removing the second photoresist to expose the first channel, the second channel, and the first chamber; and (c) sealing the electrode-supporting region and the buffer supporting region, by: (i) adhering a membrane layer to the exposed surface of the substrate to cover the first channel, the second channel, and the first chamber.

In some embodiments, the substrate is glass.

In some embodiments, the method further includes, before step (a)iv), coating the surface of the substrate with silicone dioxide, wherein said coating protects the conductive material from further patterning.

In some embodiments, the method further includes, before step (a)iv), coating the surface of the substrate with thicker silicone dioxide, wherein said coating protects the conductive material from further patterning.

In some embodiments, the conductive material is an electrically conductive material.

In some embodiments, the electrically conductive material is silver.

In some embodiments, the method includes sealing the electrode-supporting region and the network of microchannels further comprises coating the membrane layer with a sealing material. In some embodiments, the sealing material is a polymer.

In some embodiments, the polymer is selected from the group consisting of: polymethylmethacrylate (PMMA), polyethylene terephthalate (PETE), polycarbonate, and Polydimethylsiloxane (PDMS).

In some embodiments, the sealing material is Polydimethylsiloxane (PDMS).

In some embodiments, the method further comprises patterning the sealing material.

In some embodiments, sealing the electrode-supporting region and the buffer-supporting region further comprises bonding a patterned sealing component onto the membrane layer.

In some embodiments, the method further comprises breaking the membrane layer to expose the first microchannel and the second microchannel.

In some embodiments, the method further comprises adding a buffer to the first microchannel and the second microchannel.

In some embodiments, the method further comprises sealing the first channel and the second channel after adding the buffer, with a sealing material.

In some embodiments, the first microchannel and the second microchannel each have a depth that is greater than the first channel and the second channel.

In some embodiments, the first chamber is always sealed with the membrane layer.

In some embodiments, the first channel and the second channel comprise one or more electrodes. In some embodiments, the first chamber comprises a ground electrode.

In some embodiments, the first channel and the second channel comprise one or more electrodes and a buffer.

In some embodiments, the first microchannel and the second microchannel comprise a buffer.

III. Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention may also relate to a product that is produced by a computing process described herein. Such a product may include information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

1. A nanopore array device for multiplexed assays, comprising:
a chip comprising an array of nanopore components, wherein each nanopore component comprises:
(a) a first pore positioned between, and fluidically connecting, a chamber and a first fluidic channel;
(b) a second pore positioned between, and fluidically connecting, the chamber and a second fluidic channel;
wherein the first pore and the second pore are spaced apart from each other by a distance;
(c) one or more electrodes positioned within the first and second fluidic channels,
wherein the one or more electrodes are configured to apply a first voltage across the first pore, and a second voltage across the second pore; and
(d) one or more sensors configured to detect:
a current measurement that detects capture and partial or full translocation of the molecule into and through the first pore; and
a current measurement that detects capture and partial or full translocation of the molecule into and through the second pore.

2. (canceled)

3. The device of claim 1, wherein the one or more electrodes positioned within the first and second fluidic channels and the chamber is connected to one or more of:
an application-specific integrated circuit (ASIC), a field programmable gate array (FPGA), a microprocessor, and a signal processor.

4-5. (canceled)

6. The device of claim 1, wherein the sensor is configured to detect:
a voltage between the said electrode within the first fluidic channel and said electrode within the chamber for each nanopore component within the array simultaneously; and
a voltage between the said electrode within the second fluidic channel and said electrode within the chamber for each nanopore component within the array simultaneously.

7. The device of claim 1, wherein the device comprises a processor configured to:
determine from the sensor, the simultaneous presence of the molecule in both pores, and responsive to that determination, to adjust one or more of the first and second voltages to produce a first force and an opposing second force acting on the molecule,
wherein the first and second forces control the direction and speed of the molecule translocating through the first and second pores.

8-34. (canceled)

35. The device of claim 1, wherein the first fluidic channel and the second fluidic channel are V-shaped and have openings on either end of the V-shape, wherein the V-shapes of the first and second fluidic channels arranged on the chip opposite one another with points of the V-shapes being adjacent to each other, and wherein the first nanopore is positioned at the point of the V-shape of the first fluidic channel and the second nanopore is positioned at the point of the V-shape of the second fluidic channel.

36. (canceled)

37. A nanopore system for performing a multiplexed assay, comprising:
a chip comprising an array of nanopore components, wherein each nanopore component comprises:
(a) a first pore positioned between, and fluidically connecting, a chamber and a first fluidic channel;
(b) a second pore positioned between, and fluidically connecting, the chamber and a second fluidic channel;
wherein the first pore and the second pore are spaced apart from each other by a distance;
(c) one or more electrodes positioned within the first and second fluidic channels, wherein the one or more electrodes are configured to apply a first voltage across the first pore, and a second voltage across the second pore; and
(d) a sensor subsystem configured to detect:
a current measurement that detects capture and partial or full translocation of the molecule into and through the first pore; and
a current measurement that detects capture and partial or full translocation of the molecule into and through the second pore.

38. (canceled)

39. The system of claim 37, wherein the cell is a single cell.

40. The system of claim 39, wherein the single cell is loaded:
in the first fluidic channel, the second fluidic channel, or the chamber of the nanopore component,
at an opening of the first fluidic channel or an opening of the second fluidic channel ranges,
at an inlet comprising a first opening on one end of the first fluidic channel,
at an inlet comprising a first opening on one end of the second fluidic channel,
at an inlet comprising a first opening on one end of the second fluidic channel, or
at an inlet comprising a first opening of the chamber.

41-44. (canceled)

45. The system of claim 37, wherein the cell is selected from: a neuron, a muscle cell, a cardiac cell, or an oocyte.

46-47. (canceled)

48. The system of claim 37, wherein the sensor subsystem comprises a first sensor capable of identifying the presence of the molecule in the first pore, and a second sensor capable of identifying the presence of the molecule in the second pore.

49-50. (canceled)

51. The system of claim 35, wherein the system further comprises a processor and a computer-readable medium, comprising instructions that cause the processor to control the array of nanopore components as molecules translocate into and through the first and second pores of each nanopore component.

52. The system of claim 51, wherein the processor comprises one or more of: an application-specific integrated circuit (ASIC), a field programmable gate array (FPGA), a microprocessor, and a signal processor.

53. The system of claim 51, wherein the processor is connected to the one or more electrodes of the first and second fluidic channels and the one or more electrodes of the chamber.

54. (canceled)

55. The system of claim 37, wherein the assay is performed on each of the nanopore components to evaluate ion conductance of each molecule in the nanopore components.

56. The system of claim 37, wherein the sensor is configured to detect:
a voltage between the said electrode within the first fluidic channel and said electrode within the chamber for each nanopore component within the array simultaneously; and
a voltage between the said electrode within the second fluidic channel and said electrode within the chamber for each nanopore component within the array simultaneously.

57. The system of claim 37, wherein the device comprises a processor configured to:
determine from the sensor, the simultaneous presence of the molecule in both pores, and responsive to that determination, to
adjust one or more of the first and second voltages to produce a first force and an opposing second force acting on the molecule,
wherein the first and second forces control the direction and speed of the molecule translocating through the first and second pores.

58-61. (canceled)

62. The system of claim 37, wherein the distance between an outermost edge or opening of the first fluidic channel and an outermost edge or opening of the second fluidic channel ranges from 200 μm to 5 mm.

63. The system of claim 37, wherein the first fluidic channel has an inlet comprising a first opening on one end of the first fluidic channel and an outlet comprising a second opening on an opposite end of the first fluidic channel, and wherein the second fluidic channel has an inlet comprising a first opening at one end of the second fluidic channel and an outlet comprising a second opening on an opposite end of the second fluidic channel.

64-65. (canceled)

66. The system of claim 37, wherein the distance between an outermost edge of the first opening of the first fluidic channel and an outermost edge of the first opening of the second fluidic channel ranges from 100 μm to 500 μm, and wherein the distance between an outermost edge of the second opening of the first fluidic channel and an outermost edge of the second opening of the second fluidic channel ranges from 100 μm to 500 μm.

67-75. (canceled)

76. The system of claim 37, wherein the first and second pores have a diameter ranging from about 0.5 nm to about 200 nm, and wherein the length of the first and second fluidic channel ranges from about 0.05 mm to about 4 mm.

77-84. (canceled)

85. The system of claim 37 wherein the first fluidic channel and the second fluidic channel have a V-shape, and wherein the first fluidic channel and the second fluidic channel have openings on either end of the V-shape, wherein the V-shapes of the first and second fluidic channels arranged on the chip opposite one another with points of the V-shapes being adjacent to each other, and wherein the first nanopore is positioned at the point of the V-shape of the first fluidic channel and the second nanopore is positioned at the point of the V-shape of the second fluidic channel.

86. The system of claim 37, wherein the one or more electrodes are connected to a power supply configured to provide a first voltage between the first fluidic channel and the chamber, and provide a second voltage between the chamber and the second fluidic channel.

87. A method for controlling an array of nanopore components on a nanopore chip, the method comprising the steps of:
(a) performing multiplexed assays on a chip comprising an array of nanopore components, wherein each nanopore component comprises:
a first pore positioned between, and fluidically connecting, a chamber and a first fluidic channel;
a second pore positioned between, and fluidically connecting, the chamber and a second fluidic channel;
wherein the first pore and the second pore are spaced apart from each other by a distance;
one or more electrodes positioned within the first and second fluidic channels, wherein the one or more electrodes are configured to apply a first voltage across the first pore, and a second voltage across the second pore; and
one or more sensors configured to detect:
a current measurement that detects capture and partial or full translocation of the molecule into and through the first pore; and
a current measurement that detects capture and partial or full translocation of the molecule into and through the second pore.
(b) controlling the array of nanopore components with a processor and a computer-readable medium comprising instructions, that cause the processor to:
control the movement of charged molecules through the first and second pore in the array of nanopore components simultaneously, wherein each charged molecule translocates into and through the first and second pore of a single nanopore component on the array.

88-111. (canceled)

112. A method of fabricating a nanopore array device, the method comprising:
(a) generating an electrode-supporting region, by:
(i) depositing a first photoresist onto a substrate,
(ii) patterning and etching a first channel, a second channel, and a first chamber onto the substrate,
(iii) coating the entire surface of the substrate with a conductive material, and
(iv) removing the first photo resist and conductive material on the surface of the substrate outside of the first channel, the second channel, and the first chamber;
(b) generating a network of microchannels to form a buffer-supporting region in communication with first channel and the second channel, by:
(i) depositing a second photoresist on the surface of the substrate to form a partially protected region of the first channel and the second channel, and to form a completely protected region of the first chamber, and
(ii) patterning:
a first microchannel that partially overlaps with the first channel, and
a second microchannel that partially overlaps with the second channel, and
(c) removing the second photoresist to expose the first channel, the second channel, and the first chamber; and
(d) sealing the electrode-supporting region and the buffer supporting region, by:
(i) adhering a membrane layer to the exposed surface of the substrate to cover the first channel, the second channel, and the first chamber.

113-132. (canceled)
